(19) United States
(12) Patent Application Publication
(10) Pub. No.: US 2026/0144941 A1
(43) Pub. Date: May 28, 2026

(54) INJECTION ASSEMBLY

(71) Applicant: SHL Medical AG, Zug (CH)

(72) Inventors: Martin Michael Coyne, III, Boonton, NJ (US); Alejandra Linares Martinez, Boonton, NJ (US); Christopher James Franzese, Boonton, NJ (US); Jacob Leon Turetsky, Norwalk, CT (US); Yukiko Naoi, Norwalk, CT (US); Jeffrey Kapec, Norwalk, CT (US); Joshua Thomas Hartl, Baltimore, MD (US); Chester Boyd Larrow, Baltimore, MD (US); Florence Olivia Stevenson, Baltimore, MD (US); Thomas Daniel James, Baltimore, MD (US); Katherine Ford Brigham, Boonton, NJ (US); Madeleine Hayden Anderson, Boonton, NJ (US); Amy Catherine Rinaldi, Boonton, NJ (US); James Thomas Hawthorne, Boonton, NJ (US); Lukas Bannwart, Zug (CH)

(21) Appl. No.: 19/104,764

(22) PCT Filed: Aug. 21, 2023
(86) PCT No.: PCT/EP2023/072949

§ 371 Date: Jan. 9, 2026

Related U.S. Application Data
(60) Provisional application No. 63/399,743, filed on Aug. 22, 2022.
(60) Provisional application No. 63/451,699, filed on Mar. 13, 2023.

(30) Foreign Application Priority Data
Sep. 30, 2022 (EP) 22199270.4

(51) Int. Cl. A61M 5/42; A61M 5/14; A61M 5/142

(52) U.S. Cl. CPC A61M 5/427; A61M 5/1407; A61M 5/14248; A61M 2005/14252; A61M 2202/0007; A61M 2205/583; A61M 2209/088; A61M 2210/04

(57) ABSTRACT

The present disclosure provides an injection assembly for medicament administration with a plurality of needles, the injection assembly comprising: a first needle for a first predefined medicament administration, a second needle for a second predefined medicament administration, and a needle spacer arrangement configured to be removably attached to the skin of a user, the needle spacer arrangement being configured to provide a spacing between the first needle and the second needle wherein the spacing is sized such that medicament administered by the first needle and the second needle are prevented from mixing subcutaneously and/or is larger than a diameter of an expected first skin wheal formed subcutaneously as a result of the first predefined medicament administration and a diameter of an expected second skin wheal formed subcutaneously as a result of the second predefined medicament administration.

### CROSS-REFERENCE TO RELATED APPLICATIONS

The present application is a U.S. National Phase Application pursuant to 35 U.S.C. § 371 of International Application No. PCT/EP2023/072949 filed Aug. 21, 2023, which claims priority to (i) U.S. Provisional Application No. 63/399,743 filed Aug. 22, 2022, (ii) EP patent application Ser. No. 22/199,270.4 filed Sep. 30, 2022, and (iii) U.S. Provisional Application No. 63/451,699 filed Mar. 13, 2023. The entire disclosure contents of these applications are herewith incorporated by reference into the present application.

### TECHNICAL FIELD

The present disclosure generally relates to medical devices for medicament administration.

### BACKGROUND

Infusion and injection are commonplace medical procedures used to deliver a wide variety of therapeutic medicines of interest for a variety of diseases. “Infusion,” “injection,” and “administration” may be used interchangeably, taking place by subcutaneous (SC), intramuscular (IM), intravenous (IV), enteral, or other routes, also terms used interchangeably. Administration route is based on a specific medication's pharmacokinetic (PK) profile, formulation components, approved regulatory labelling, individual clinical judgment, or clinical necessity.

The SC route is frequently used for administration of smaller volumes using prefilled syringes and autoinjectors. Biologic medicines are frequently administered via the SC route with these devices. SC administration is generally considered less invasive and more straightforward for patients, particularly compared to IV administration. Additionally, as physiologic uptake of medication is slower via the SC route, there is potential for improved tolerability compared to IV administration.

Given these significant advantages in safety, tolerability, and convenience, the pharmaceutical industry has invested heavily in transitioning formulations from IV to SC administration and medication administration from the clinic to the home setting. However, progress in large-volume SC (LVSC) administration (administrating medicament more than 3 ml subcutaneously) poses several issues that are related to the tolerability of a large volume of a single medication and the implications of this on pharmacokinetics when numerous LVSC medications are given sequentially or concurrently.

Certain biologic medications must be given in larger volumes that exceed the capacity of prefilled syringes and autoinjectors. For these medications, a larger volume device is used, such as an on-body injector (OBI, also known as a large volume infuser, “LVI,” or bolus injector) or pump and SC needle set. Such SC needle sets include a hollow-bore needle with sharpened point for percutaneous access to patient anatomy, and a tubing set for connection to the pump. Each needle may be used to deliver a specific volume of medication subcutaneously, limited by patient anatomy. For some medications of moderate volume infused relatively slowly, a single needle may suffice, as in the case of OBI devices. For other medications of comparatively larger volume, anywhere from 2-8 needles may be used, the number of needles generally increasing with increasing total medication volume and/or increasing infusion rate. Multiple needles may be connected with a splitter which allows a single medication reservoir, driven by a single pump, to split medication flow between the medication administration needles.

The required number of medication administration needles must be inserted by the health care provider, by the patient themselves, or by a lay (i.e., clinically untrained) caregiver. They must contend with appropriate anatomical placement, avoiding scarred areas, areas with broken skin, and certain aspects of anatomy, such as the umbilicus. Additionally, tubing sets used to connect each of the needle sets to the splitters or the medication reservoir must be managed and may be easily tangled. It is often difficult to attach one needle set when it becomes tangled with another, making the process frustrating and confusing for users lacking clinical training, such as the patients themselves. Particularly for untrained or non-clinical users, this application process may be awkward and confusing.

Additional complexity is presented when multiple medications are given. Several studies outline the physiologic effects of injecting larger volumes into the SC space. These data indicate that the size of SC medication depots, and corresponding skin “wheal” or prominence, depend on injection volume, rate, viscosity, and inter-individual variability, among other potential factors. Notably, prior clinical studies reported in the peer-reviewed literature have focused on injection of a single medication. When multiple medications are present, it is desirable for a clinician or pharmaceutical company to administer multiple medications to multiple injection sites, either in sequence or concurrently, while preventing inadvertent mixing or diffusion of medications between sites during administration. The underlying science of this phenomenon may not be predictable to pharmaceutical companies a priori or intuitive to those lacking clinical training, such as the patient themselves or a lay caregiver. However, this phenomenon is strongly dependent on needle placement, and particularly, relative placement of the needles upon the skin.

### SUMMARY

In view of the above, there is a need for improved solutions to ensure separations of multiple medicaments at their respective injection sites, or administration of a single medication across multiple injection sites, in a controlled, predictable manner.

There is hence provided an injection assembly for medicament administration with a plurality of needles, the injection assembly comprising: a first needle for a first predefined medicament administration, a second needle for a second predefined medicament administration, and a needle spacer arrangement configured to be removably attached to the skin of a user, the needle spacer arrangement being configured to provide a spacing between the first needle and the second needle, wherein the spacing is predetermined based on at least one of characteristics of the medicament, characteristics of infusion parameters for each medicament, physiologic parameters, or combinations thereof.

Thus, the spacing is sized such that medicament administered by the first needle and the second needle are prevented from mixing subcutaneously and/or is larger than a diameter of an expected first skin wheal formed subcutaneously as a result of the first predefined medicament administration and a diameter of an expected second skin wheal formed subcutaneously as a result of the second predefined medicament administration.

The injection assembly thus can provide for multiple simultaneous injections while ensuring proper distancing 0f the needles from each other. This generally means that a safety distance between different medicaments can be set. Thus, the spacing between the first needle and the second needle that is provided by the needle spacer arrangement can be designed based on the predefined medicament administrations.

A “skin wheal” is the subcutaneous accumulation of liquid medicament that in the course of medicament administration has spread in a substantially radial manner outwardly from the needle around the injection site, and which the body is not able to immediately absorb. As the skin wheal overlapping issue is a common problem for large molecule and/or high-volume medicament administration; in other words, small volume and/or small molecule medicament administration, for example, an insulin and/or glucagon injection will not have such problem as the insulin and/or glucagon injection usually is small volume injection, as an insulin vials and pens commonly have a concentration of 100 units in 1 mL (100 units/mL), which is called U-100, and the patients usually are instructed to take 1-3 units of insulin for each injection, namely that the insulin injection is usually small volume such as 0.01-0.03 mL. Furthermore, the molecular weight of insulin is 5734 daltons that is usually considered as small molecule medicament and is less likely to cause a skin wheal as the medicament is easy to be absorbed for most people.

Thus, the injection assembly disclosed herein is configured to be used with high-volume medicament administration. For example, the amount of the first predefined medicament administration is above 1 ml, preferably above 3 ml; the amount of the second predefined medicament administration is above 1 ml, preferably above 3 ml. Furthermore, the sum of the amount of the first predefined medicament administration and the amount of the second predefined medicament administration is above 10 ml, preferably, above 25 ml. Alternatively, the injection assembly is configured to be used to deliver large molecule medicament. As biologic medications (proteins with a therapeutic effect) are extracted or adapted from living organisms, as by genetic engineering, and have comparatively higher molecular weights 10-1000 kilodaltons (kDa). For example, the molecular weight of the first predefined medicament is above 10 kDa and/or the molecular weight of the second predefined medicament is above 10 kDa.

The needle spacer arrangement as disclosed herein can provide separation of subcutaneous depots based on characteristics of the medicament, infusion parameters for each medicament, physiologic parameters, or combinations thereof. Combinations of these factors may affect the shape, size, and rate of growth of the subcutaneous depot. The subcutaneous depot, in turn, may affect pharmacokinetics of the medicament(s) being injected with the apparatus.

This is particularly important when different medications are used, as is common in an oncology regimen. Different sequences of small and large molecules may be administered, and it is clinically important to prevent the subcutaneous depots under the skin from commingling or overlapping to achieve proper therapeutic effect.

Characteristics of the medicament may include whether the medicament is a small or large molecule, concentration, viscosity, the presence of dispersion enhancers, such as hyaluronidase. Characteristics infusion parameters may include the infusion rate, volume, viscosity, and non-Newtonian behaviours (such as shear thinning or thickening). Infusion parameters may also relate to the device used to infuse medication into the skin, such as a steel needle or soft cannula, injection cannula length or inner diameter, or materials of construction. It is generally desirable to infuse medication at the highest rate possible, subject to physiologic limitations. Taken in conjunction with physiologic limitations, these factors affect the maximum possible delivery rate possible with the medicament delivery device. Physiologic parameters may include absorption, bioavailability, subcutaneous thickness, skin turgor, tissue backpressure in response to infusion, among other factors.

While presented here as independent factors, many factors may be correlated. With knowledge of the characteristics of the medicament (e.g., through laboratory characterization and simulated injections), infusion parameters for each medicament (e.g., through human testing and SC depot characterization), and physiologic parameters (e.g., through pharmacokinetic and/or pharmacodynamic in-human studies or in silico modeling), the design parameters for each needle spacer may be selected to avoid undesired commingling or overlapping. Different factors may be of interest for each medicament.

The size of the spacing is predetermined, based on at least one of the viscosities of first and the second predefined medicament, physiologic statistic data, the predefined administration rate of the first and the second predefined medicament, and the amount of the first and the second predefined medicament.

With predefined medicament administration is meant that a predefined type of drug/medicament is to be administered at a predefined rate, for a predefined amount of time. The target patient group is also predefined. This generally means that the expected size of a skin wheal is relatively well-defined especially if the skin wheal size variation based on the absorption capability of various users has been considered.

Each needle disclosed herein may for example be one of a right angle needle, a straight needle, or a soft cannula, or a soft cannula with an insertion needle configured to penetrate the skin of the user. It should be noted that most of the insertion needles used with the soft cannula will be removed after penetration, thus, only the soft cannula remains in user's body during the medicament delivery operation.

In the present disclosure, when the term “distal direction” is used, this refers to the direction pointing away from the dose delivery site during use of the medicament delivery device. When the term “distal part/end” is used, this refers to the part/end of the delivery device, or the parts/ends of the members thereof, which under use of the medicament delivery device is/are located furthest away from the dose delivery site.

Correspondingly, when the term “proximal direction” is used, this refers to the direction pointing towards the dose delivery site during use of the medicament delivery device.

When the term “proximal part/end” is used, this refers to the part/end of the delivery device, or the parts/ends of the members thereof, which under use of the medicament delivery device is/are located closest to the dose delivery site.

Further, the term “longitudinal”, “longitudinally”, “axially” or “axial” refer to a direction extending from the proximal end to the distal end, typically along the device or components thereof in the direction of the longest extension of the device and/or component.

Similarly, the terms “transverse”, “transversal” and “transversally” refer to a direction generally perpendicular to the longitudinal direction.

Further, the terms “circumference”, “circumferential”, or “circumferentially” refer to a circumference or a circumferential direction relative to an axis, typically a central axis extending in the direction of the longest extension of the device and/or component.

Similarly, “radial” or “radially” refer to a direction extending radially relative to the axis, and “rotation”, “rotational” and “rotationally” refer to rotation relative to the axis.

According to one embodiment the needle spacer arrangement comprises a first needle spacer element and a second needle spacer element physically separate from the first needle spacer element.

The first needle spacer element and the second needle spacer element may have the same size or have different sizes.

According to one embodiment the first needle spacer element has a first dimension, and the second needle spacer element has a second dimension. The first needle spacer element comprises a portion attached to the first needle and the second needle spacer element comprises a portion attached to the second needle. The first dimension and the second dimension are arranged such that when the first needle spacer element is adjacent to the second needle spacer element on one surface, the first needle and the second needle are spaced apart from one another at a distance at which the medicament administered by the first needle and the second needle are prevented from mixing subcutaneously and/or is larger than a diameter of an expected first skin wheal formed subcutaneously as a result of the first predefined medicament administration and a diameter of an expected second skin wheal formed subcutaneously as a result of the second predefined medicament administration.

The first dimension can be the same as the second dimension, or the first dimension can be different from the second dimension.

The first needle spacer element may for example be circular, elliptic, or have another shape, such as polygonal.

The second needle spacer element may for example be circular, elliptic, or have another shape, such as polygonal.

According to one embodiment the first needle spacer element is circular, and the first dimension is a first diameter.

According to one embodiment the second needle spacer element is circular, and the second dimension is a second diameter.

According to one embodiment the first needle spacer element comprises a main portion configured to be attached to the skin of the user, and a side wall extending from the main portion in the direction perpendicular to the main portion such that when the second needle spacer element is overlapped with the first needle spacer element, the second needle spacer element is blocked from attaching to the skin of the user.

Alternatively, or additionally, according to one embodiment the first needle spacer element comprises a wearable feature configured to be worn by the user such that a main portion of the first needle spacer element is attached to the skin of the user, and a side wall extending from the main portion in the direction perpendicular to the main portion such that when the second needle spacer element is overlapped with the first needle spacer element, the second needle spacer element is blocked from attaching to the skin of the user.

According to one embodiment, the first needle spacer element and the second needle spacer element are magnetic. The first needle spacer element is configured to magnetically repel the second needle spacer element when the second needle spacer element is arranged within a predetermined distance from the first needle spacer. This may facilitate placement of the needle spacer elements.

According to one embodiment the first needle is mounted to the first needle spacer element and the second needle is mounted to the second needle spacer element.

According to one embodiment the first needle is mounted to the main portion of the first needle spacer element and the second needle is mounted to the main portion of the second needle spacer element.

According to one embodiment the needle spacer arrangement comprises a needle guide configured to physically separate the first needle from the second needle.

The needle guide can be used with the first needle and the second needle directly. In this embodiment, the needle guide is configured to physically separate the first needle from the second needle directly. Alternatively, the needle guide can be used with the first needle spacer element and the second needle spacer element. In this embodiment, the needle guide is configured to physically separate the first needle from the second needle by physically separating the first needle spacer element from the second needle spacer element.

Furthermore, the needle guide may be configured to guide the placement of the first and the second needle on the skin of the user, for example, the needle guide comprises an opening and/or a guide wall to guide the placement of the needle, either by interaction with the first and second needle spacer element or by interaction with the first and second needle.

According to one embodiment the needle spacer arrangement comprises the needle guide having the guide wall being an outer contour which forms a first guide surface and a second guide surface, the first guide surface being adapted to an outer contour of the first needle spacer element for positioning the first needle spacer element relative to the needle guide, and the second guide surface being adapted to an outer contour of the second needle spacer element for positioning the second needle spacer element relative to the needle guide.

The needle guide may have an inner surface configured to be removably attached to a user's body. In this example, the needle guide comprises an outer surface opposite to the inner surface, namely that the outer surface of the needle guide is facing away from the user's body during use. The outer contour may form a surface that is at a right angle with the inner surface, or the outer contour may be at an angle other than 90 degrees. For example, the outer contour may form a bevelled surface relative to the inner surface.

According to one embodiment the needle spacer arrangement comprises the needle guide. The needle guide may be configured to be releasably attached to the skin of the user and configured to physically separate the first needle spacer element from the second needle spacer element. The guide wall of the needle guide is an outer wall extending in the direction parallel to the needle. In one example, the outer wall forms the first guide surface and the second guide surface. Alternatively, the needle guide comprises at least two outer walls extending in the direction parallel to the needle; in this example, the at least two outer walls form the first guide surface and the second guide surface respectively.

According to one embodiment, a magnetic guide can be provided. In this embodiment, the first needle spacer element and the second needle spacer element (or portions thereof) may be magnetic and may be optionally configured with different (i.e., attracting or repelling) combinations of polarities (i.e, north and south poles). The first guide surface may be magnetic and configured to attract the first needle spacer element with a certain orientation and to repel the second needle spacer element. The second guide surface may be magnetic and configured to attract the second needle spacer element with a certain orientation and to repel the first needle spacer element. This may facilitate placement of the needle spacer elements.

Alternatively, or additionally, according to another embodiment, a texture guide can be provided. For example, the first guide surface and the second guide surface can be formed as smooth surfaces; and a portion of the first needle spacer element can be formed with a smooth surface, and the rest portions of the first needle spacer element can be formed with a rough surface; similarly, a portion of the second needle spacer element can be formed with a smooth surface, and the rest portions of the second needle spacer element can be formed with a rough surface. In this example, the user can be guided to place the smooth surfaces of the needle spacer elements adjacent to the smooth surface of the needle guide. In another example, a structural guide can be provided by different shapes. For example, the first guide surface can be a concave surface configured to match with a needle spacer element with a convex surface; and the second guide surface can be convex surface configured to match with a needle spacer element with a concave surface.

According to one embodiment each of the first guide surface and the second guide surface is noncongruent with a respective one of the first needle spacer element and the second needle spacer element.

Thus, the first needle spacer element and the second needle spacer element may not be completely mating. Instead, tangential contact signals a user that the needle spacer elements are properly placed. This configuration requires less precise placement during needle application than if the first guide surface and the second guide surface is configured to mate with a respective one of the first needle spacer element and the second needle spacer element.

According to one embodiment the first guide surface (or portions thereof) is colour matched with the first needle spacer element, and the second guide surface (or portions thereof) is colour matched with the second needle spacer element.

Colour cues are thereby provided to facilitate the placement of the first and second needle spacer element. The colour cues can be selected that such set of colours that can be reliably identified and distinguished between by those with or without forms of colour-blindness.

Furthermore, as the medicament delivery devices configured to deliver large volume of medicament to a user are usually placed remotely relative to the medicament delivery sites. For example, an infusion pump may be placed 1-2 meters away from the user's skin. In this case, the user usually is instructed to place the first and the second needle to the medicament delivery sites with the first and the second needle spacer element respectively before connecting one or more tubes between the needles and the medicament delivery device. In this example, the user can be instructed to mark the needle spacer elements with the colour that is matched to the colour cues of the guide surfaces by, e.g., colouring the needle spacer elements with pens or stickers. Thus, after the needle guide is removed, the user can be further instructed to connect the tubes between the medicament delivery device and the needles in a correct way. For example, the user can be instructed to connect the blue tube to a needle spacer element that is coloured with blue.

According to one embodiment the needle spacer arrangement comprises a needle guide physically separate from the first needle spacer element and from the second needle spacer element, the needle guide having a first opening configured to receive the first needle spacer element, and a second opening configured to receive the second needle spacer element. In this embodiment, the first guide surface is defined by the contour of the first opening; and the second guide surface is defined by the contour of the second opening.

In one example where the needle guide has the inner surface configured to be removably attached to a user's body. Each of the first and second opening may be defined by a surface that is at a right angle with the inner surface, or the outer surface may be at an angle other than 90 degrees. For example, the surface may form a bevelled surface relative to the inner surface. Similar to the above-mentioned embodiments, a magnet guide or a structural guide can be provided between the openings of the needle guide and the needle spacer elements.

According to one embodiment the needle spacer arrangement comprises the needle guide. The needle guide is configured to be releasably attached to the skin of the user and configured to physically separate the first needle spacer element from the second needle spacer element. The needle guide extends in the transverse direction to the needle(s). The needle guide has the first opening extending through the needle guide in the direction parallel to the needle(s), and the second opening extending through the needle guide in the direction parallel to the needle(s). According to one embodiment the needle guide comprises a third opening configured to receive the first needle spacer element, and a fourth opening configured to receive the second needle spacer element.

The needle guide comprises the third opening extending through the needle guide in the direction parallel to the needle(s), and a fourth opening extending through the needle guide in the direction parallel to the needle(s).

It should be noted that, in the example where the injection assembly comprises the first and the second needle spacer elements, when the needle guide comprises multiple openings, e.g., the first opening, the second opening, the third opening, and the fourth opening, each corresponding guide surfaces, e.g., the first guide surface, and the second guide surface, is defined by the contour of each opening respectively.

According to one embodiment the needle spacer arrangement comprises a needle guide which is generally I-shaped, wherein the first needle is arranged at a first end of the I and the second needle is arranged at a second end of the I, opposite to the first end, or wherein the needle guide is generally X-shaped, wherein the first needle is arranged in an end of an arm of the X and the second needle is arranged in an end of a leg of the X, or wherein the needle guide is generally Y-shaped, wherein the first needle is arranged in an end of a leg or arm of the Y, and the second needle is arranged an end of another arm of the Y.

According to one embodiment the needle guide comprises a location through-opening defining an anatomical reference point.

A user may for example find the anatomic reference point using a finger through the location through-opening. Needles need not be at regular angularly spaced intervals from the anatomic reference point, and may be provided symmetrically along an axis, asymmetrically along an axis, or combinations thereof. When multiple needles are used for large volume subcutaneous delivery, two, three, or four needles may be desirable, oriented radially from each other and centred around the anatomical reference point.

According to one embodiment the needle guide is shaped to be attached to the abdomen, wherein the anatomical reference point is the umbilicus, or to a leg, wherein the anatomical reference point is the knee.

According to one embodiment the needle guide is shaped to be attached to the abdomen, wherein the anatomical reference point is the umbilicus, or to a leg, wherein the anatomical reference point is the knee. For example, the guide is intended to be used on the abdomen and the elongated oval on the thigh. While other shapes for each guide are obviously possible, the size and shape of each guide should be such that a user receives negative cues about placement of that guide in the opposite location. For example, placing the abdomen guide on the top of the thigh may wrap undesirably around the circumference of the leg or extend over the bend of the knee, both indicating to the user that this is not the correct application.

The shape of the needle guide may for example suggest anatomic placement on the abdomen and will in this case clearly not fit a thigh or back-of-arm injection site. Inclusion or omission of the location through-opening may also provide a strong form-based signal to a user of the injection assembly. Inclusion of a locating through-opening may be advantageous for some anatomical reference points, such as the umbilicus, but may be undesirable for other sites, such as the upper thigh or back of arm. In these latter instances, the needle guide may be placed on the skin at the desired anatomical location and may be more accurately located by other features in lieu of a location through-opening, such as a notch for the shoulder or for the top of the knee. Other features may be provided based on the desired site and patient anatomy. Features may also be suggestive of (i.e., encourage) a desired anatomic site, or alternatively, may be contoured to discourage an undesired anatomic site.

According to one embodiment the needle guide has a symmetrical shape with respect to the location through-opening, or an asymmetrical shape with respect to the location through-opening.

According to one embodiment the needle guide comprises an adhesive surface configured to be removably attached to the skin of the user.

According to one embodiment the needle guide comprises an adhesive material arranged on the inner surface of the needle guide configured to be removably attached to the skin of the user.

According to one embodiment the needle guide comprises multiple layers of adhesive materials arranged on the inner surface of the needle guide configured to be removably attached to the skin of the user.

According to one embodiment the needle guide is reusable. In this example, a layer of the multiple layers of adhesive materials is configured to be removably attached to the skin of the user, and the layer that is attached to the skin of the user is configured to be removed from the needle guide after use of the needle assembly.

Alternatively, according to one embodiment the needle guide is configured to be removably attached to the skin of the user by being held by the user onto the skin.

According to one embodiment the needle guide has the inner surface and the outer surface opposite to the inner surface, wherein the outer surface has an instructional text and reversed instructional text describing an anatomical site of the anatomical reference point.

The instructional text for using the injection assembly may thus be both right-reading and reverse reading/mirrored. The reversed instructional text facilitates reading the user instructions when using a mirror to correctly place the needle guide on the skin. Such may be the case, for instance, when users are applying the injection assembly to their own body, or when a caregiver is attaching the injection assembly to a user of the medicament delivery device using a mirror.

The needle guide according to any example disclosed herein may be a flexible sheath. The needle guide may for example comprise a polymer material, such as an elastomer material, e.g., rubber or silicone rubber, a biopolymer material such as cellulose, a compliant open or closed-cell foam material, or other suitable material.

According to one embodiment the first and/or second needle spacer elements and/or the needle guide as mentioned above may comprise a needle mount configured to be releasably attached to a counter needle mount to which the needle is connected. Alternatively, or additionally, in one example where the needle spacer arrangement comprises the needle spacer elements and the needle guide and the needles are configured to be attached to the needle guide via the needle spacer elements, the needle guide may comprise needle mounts configured to be releasably attached to counter needles mounts of the needle spacer elements.

According to one embodiment, the needle mount and the counter needle mount are configured to form a hook-and-loop fastener. In this embodiment, the needle guide and/or the needle spacer elements can be reusable.

As mentioned above, the medicament delivery device for large volume medicament delivery might be remoted to the medicament delivery sites and uses tubes to connect the needles and the medicament delivery device. Thus, there is a risk that the user might accidentally pull the tubes during use of the medicament delivery device. The fastener between the needle mount and the counter needle mount provides a safety mechanism to avoid the needles from being pulled when the user accidentally pulls the tubes. Thus, the user can be protected from injury by the needles.

There is according to a second aspect provided a method of administering medicament by means of a plurality of needles, the method comprising: step a) placing an injection assembly of the first aspect on a user, and step b) administering medicament subcutaneously simultaneously or sequentially by means of the first needle and the second needle.

According to one example the needle spacer arrangement comprises a first needle spacer element to which the first needle is mounted and a second needle spacer element physically separate from the first needle spacer element, to which the second needle is mounted, and a needle guide having an outer contour which forms a first guide surface and a second guide surface, the first guide surface being adapted to an outer contour of the first needle spacer element for positioning the first needle spacer element relative to the needle guide, and the second guide surface being adapted to an outer contour of the second needle spacer element for positioning the second needle spacer element relative to the needle guide, wherein step a) involves a1) first attaching the needle guide to the user, and a2) subsequently positioning and attaching the first needle spacer element using the first guide surface and the second needle spacer element using the second guide surface.

Alternatively, the needle guide may be provided with a plurality of openings, wherein step a) involves a1) first attaching the needle guide to the user, and a2′) arranging the first needle spacer element in one of the openings and arranging the second needle spacer element in another one of the openings.

Alternatively, the needle guide may be provided with a plurality of openings, wherein step a) involves a1) first attaching the needle guide to the user, a2″) arranging the first needle in one of the openings and arranging the second needle in another one of the openings, and a3″) at least partially attaching the first needle spacer element onto the needle guide and at least partially attaching the second needle spacer element onto the needle guide.

According to one example, the method comprises removing the needle guide from the user after step a2), prior to step b).

According to one example, the method comprises removing the needle guide from the user after step a2′), prior to step b).

According to one example, the method comprises removing the needle guide from the user after step a2″), prior to step b).

It should be noted that the needle guide can be used without first needle spacer and the sconed needle spacer. In this embodiment, when the above-mentioned method steps are carried out, instead of arranging the first needle spacer element and the second needle spacer element to the needle guide, the first needle and the second needle are directly arranged to the needle guide.

For example, when the needle guide has an outer contour which forms a first guide surface and a second guide surface, the first guide surface is adapted to an outer contour of the first needle for positioning the first needle relative to the needle guide, and the second guide surface being adapted to an outer contour of the second needle for positioning the second needle relative to the needle guide, wherein step a) involves a1) first attaching the needle guide to the user, and a2′″) subsequently positioning and attaching the first needle using the first guide surface and the second needle using the second guide surface.

Alternatively, when the needle guide is provided with a plurality of openings, wherein step a) involves a1) first attaching the needle guide to the user, and a2″″) arranging the first needle in one of the openings and arranging the second needle in another one of the openings.

According to one example, the method comprises removing the needle guide from the user after step a2′″), prior to step b).

According to one example, the method comprises removing the needle guide from the user after step a2″″), prior to step b).

Removing the needle guide from the user and leaving the first needle and the second needles removably attached to the user's skin allows freer movement of the user without interference from a portion of the needle guide during the injection process.

There is according to a third aspect provided a kit of parts comprising a plurality of needle guides, each needle guide having an outer contour which forms a first guide surface and a second guide surface, the first guide surface being adapted to an outer contour of a first needle spacer element for positioning the first needle spacer element relative to the needle guide, and the second guide surface being adapted to an outer contour of a second needle spacer element for positioning the second needle spacer element relative to the needle guide, wherein a location of the first guide surface and a location of the second guide surface is mutually different between the needle guides of the set.

The angle of the needles relative to an anatomic reference point or landmark can thereby be varied within a sequence, providing inherent site rotation between infusions.

There is according to a fourth aspect provided a kit of parts comprising a plurality of needle guides, each needle guide having a first opening and a second opening configured to receive a respective needle, wherein a location of the first opening and a location of the second opening is mutually different between the needle guides of the set.

According to one example, the needle guide comprises a location through-opening defining an anatomical reference point.

According to one example, the needle guide is shaped to be attached to the abdomen, wherein the anatomical reference point is the umbilicus, or to a leg, wherein the anatomical reference point is the knee.

According to one example, each needle spacer arrangement may be provided with a respective right reading or reversed reading indicia to indicate an injection configuration corresponding to one amongst a sequence. For example, the right reading or reversed reading indicia may be “A”, “B”, or “C”, or “Week 1”, Week 2”, or “Week 3”.

There is according to a fifth aspect provided a kit of the needle assembly comprising a needle guide, and a needle guide storage. The inner surface of the needle guide is configured to be attached to the needle guide storage.

There is according to a sixth aspect of the invention provides a needle guide for medicament administration with a plurality of needles, wherein the needle guide is configured to be removably attached to the skin of a user and is configured to provide a spacing between a first needle of the plurality of needles and a second needle of the plurality of needles, comprising: a first guide surface and a second guide surface, the first guide surface is configured to operably connected to the first needle for positioning the first needle relative to the needle guide, and the second guide surface is configured to operably connected to the second needle for positioning the second needle relative to the needle guide.

According to one example, the needle guide has an outer contour which forms the first guide surface and the second guide surface, the first guide surface being adapted to an outer contour of a first needle spacer element for positioning the first needle spacer element relative to the needle guide, and the second guide surface being adapted to an outer contour of a second needle spacer element for positioning the second needle spacer element relative to the needle guide.

According to one example, each of the first guide surface and the second guide surface is configured to mate with a respective one of the first needle spacer element and the second needle spacer element.

According to one example, each of the first guide surface and the second guide surface is noncongruent with a respective one of the first needle spacer element and the second needle spacer element.

According to one example, the first guide surface is colour matched with the first needle spacer element, and the second guide surface is colour matched with the second needle spacer element.

According to one example, the needle guide has a first opening configured to receive the first needle, and a second opening configured to receive the second needle; wherein the first guide surface is either the inner contour of the first opening or the surface around the first opening; and wherein the second guide surface is either the inner contour of the second opening or the surface around the second opening.

According to one example, the needle guide comprises a first needle mount adjacent to the first opening, and a second needle mount adjacent to the second opening; and wherein the first needle and the second needle are configured to be releasably mounted to the first and the second needle mounts of the needle guide.

According to one example, the needle guide comprises multiple openings for receiving multiple needles respectively; wherein the needle guide comprises multiple needle mounts adjacent to the multiple openings respectively; and wherein at least one needle mount of the multiple needle mounts comprises an identifying feature to differentiate the at least one needle mount from the rest of the multiple needle mounts.

According to one example, the first opening is configured to receive a first needle spacer element, and the second opening is configured to receive a second needle spacer element.

According to one example, the first needle spacer element comprises a first counter needle mount configured to be engaged with the first needle mount and the second needle spacer element comprises a second counter needle mount configured to be engaged with the second needle mount.

According to one example, the first opening is configured to receive a first needle inserter, and the second opening is configured to receive a second needle inserter.

According to one example, the first needle mount and the first counter needle mount are configured to form a hook-and-loop fastener.

According to one example, the second needle mount and the second counter needle mount are adhesive.

According to one example, needle guide comprises a location through-opening defining an anatomical reference point.

According to one example, the needle guide is shaped to be attached to the abdomen, wherein the anatomical reference point is the umbilicus, or to a leg, wherein the anatomical reference point is the knee.

According to one example, the needle guide has a symmetrical shape with respect to the location through-opening, or an asymmetrical shape with respect to the location through-opening.

According to one example, the needle guide has an inner surface and an outer surface opposite to the inner surface, wherein the outer surface has an instructional text and reversed instructional text describing an anatomical site of the anatomical reference point.

According to one example, the needle guide comprises a wearable element for being worn by the user.

According to one example, the wearable element is a belt, a shoulder stripe, a neck stripe, a garment, a leg sleeve, or a belt clip.

According to one example, the needle guide comprises a distributing hub fluidly connected to the medicament delivery device; and wherein a tube is connected to the distributing hub and at least one of the first needle and the second needle.

According to one example, the needle guide comprises tubing management hub configured to retain a tube connected to the medicament delivery device.

According to one example, the tubing management hub is attached to the needle guide.

Generally, all terms used in the claims are to be interpreted according to their ordinary meaning in the technical field, unless explicitly defined otherwise herein. All references to “a/an/the element, apparatus, component, means, etc.” are to be interpreted openly as referring to at least one instance of the element, apparatus, component, means, etc., unless explicitly stated otherwise.

### BRIEF DESCRIPTION OF THE DRAWINGS

The specific embodiments of the inventive concept will now be described, by way of example, with reference to the accompanying drawings, in which:

FIG. 1A shows an example of an injection assembly;

FIG. 1B shows when the injection assembly when provided subcutaneously at an injection site;

FIG. 2 shows another example of an injection assembly;

FIGS. 3A-3C show a sequence of use according to variation of the injection assembly in FIG. 2;

FIG. 4A depicts another example of an injection assembly provided to a user;

FIG. 4B shows a variation of the injection assembly in FIG. 4A;

FIG. 5 shows an example of a needle guide;

FIG. 6 shows an example of an injection assembly comprising medicament delivery devices;

FIG. 7 shows another example of an injection assembly comprising medicament delivery devices;

FIG. 8 shows another example of an injection assembly comprising a medicament delivery device;

FIG. 9 is another example of an injection assembly;

FIG. 10 shows the injection assembly in FIG. 9 on a user;

FIG. 11 shows another example of an injection assembly;

FIG. 12 depicts the injection assembly in FIG. 11 on a user;

FIG. 13 is yet another example of an injection assembly;

FIGS. 14A-14B show a sequence of use of the injection assembly in FIG. 13; and

FIG. 15 shows removal of a variation of the injection assembly in FIG. 12 from a user's body.

FIGS. 17-23 show other examples of the injection assembly of the invention.

FIGS. 24-26 show other examples of the needle guide of the injection assembly of the invention.

FIGS. 27A-27B show other examples of the needle and the needle guide of the injection assembly of the invention.

FIGS. 28-32 show other examples of the needle guide of the injection assembly of the invention.

FIGS. 23-38B show other examples of the needle spacer element of the injection assembly of the invention.

### DETAILED DESCRIPTION

The inventive concept will now be described more fully hereinafter with reference to the accompanying drawings, in which exemplifying embodiments are shown. The inventive concept may, however, be embodied in many different forms and should not be construed as limited to the embodiments set forth herein; rather, these embodiments are provided by way of example so that this disclosure will be thorough and complete, and will fully convey the scope of the inventive concept to those skilled in the art. Like numbers refer to like elements throughout the description.

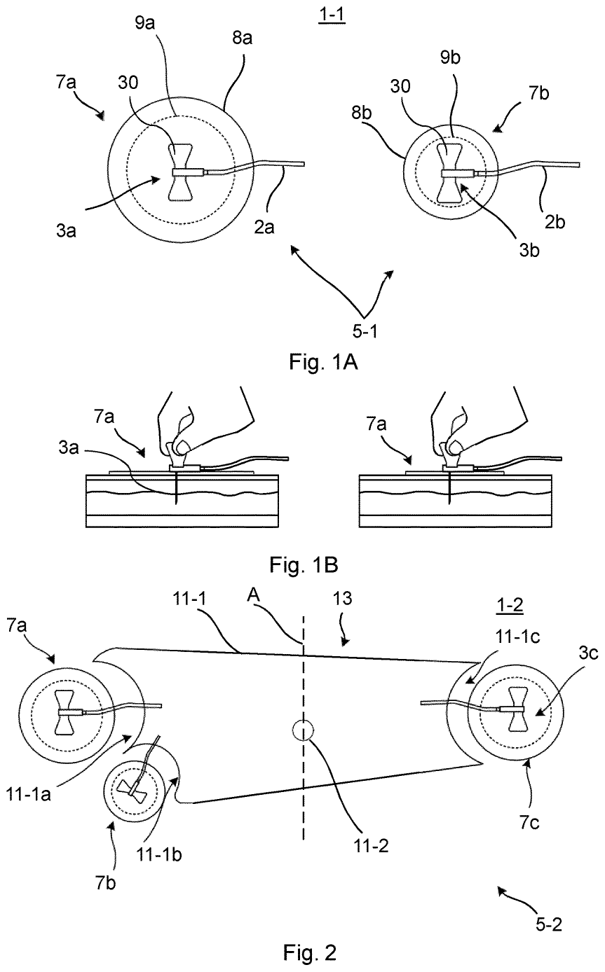

FIG. 1A shows a first example of an injection assembly according to the present disclosure.

The injection assembly 1-1 comprises a first needle 3a and a second needle 3b. The injection assembly 1-1 may optionally comprise more than two needles.

The first needle 3a is for providing a first predefined medicament administration. The second needle 3b is for providing a second predefined medicament administration.

The first needle 3a is connected to a first tube 2a; 2a′, and the second needle is connected to a second tube 2b; 2b′. The first and the second tubes 2a, 2b; 2a′, 2b′ are operably connected to a medicament delivery device (not shown). The medicament delivery device typically comprises a medicament container containing a medicament, and a pump configured to pump the medicament through at least one of the first and the second tubes 2a, 2b; 2a′, 2b′ to the needle(s) 3a, 3b. In some examples, the same medicament delivery device may provide medicament to both needles 3a, 3b, while in other examples each needle 3a, 3b receives medicament from a respective medicament delivery device. This may typically be the case if the two needles 3a, 3b are used for administering two different kinds of medicament. The injection assembly comprises the needle spacer arrangement which provides a spacing between the first needle and the second needle.

The spacing is predetermined based on characteristics of the medicament, infusion parameters for each medicament, physiologic parameters, or combinations thereof. Combinations of these factors may affect the shape, size, and rate of growth of the subcutaneous depot. The subcutaneous depot, in turn, may affect pharmacokinetics of the medicament(s) being injected with the apparatus.

Different sequences of small and large molecules may be administered, and it is clinically important to prevent the subcutaneous depots under the skin from commingling or overlapping to achieve proper therapeutic effect.

Characteristics of the medicament may include whether the medication is a small or large molecule, concentration, viscosity, the presence of dispersion enhancers, such as hyaluronidase. Characteristics infusion parameters may include the infusion rate, volume, viscosity, and non-Newtonian behaviours (such as shear thinning or thickening). Infusion parameters may also relate to the device used to infuse medication into the skin, such as a steel needle or soft cannula, injection cannula length or inner diameter, or materials of construction. It is generally desirable to infuse medication at the highest rate possible, subject to physiologic limitations. Taken in conjunction with physiologic limitations, these factors affect the maximum possible delivery rate possible with the medicament delivery device. Physiologic parameters may include absorption, bioavailability, subcutaneous thickness, skin turgor, tissue backpressure in response to infusion, among other factors.

While presented here as independent factors, many factors may be correlated. With knowledge of the characteristics of the medicament (e.g., through laboratory characterization and simulated injections), infusion parameters for each medicament (e.g., through human testing and SC depot characterization), and physiologic parameters (e.g., through pharmacokinetic and/or pharmacodynamic in-human studies or in silico modeling), the design parameters for each needle spacer may be selected to avoid undesired commingling or overlapping. Different factors may be of interest for each medication. Two examples shown in the Table 1 below.

TABLE 1
An exemplified infusion circle of an oncologic treatment

| | 1 | 2 | 3 |
|---|---|---|---|
| Illustrative | Comparatively small | Comparatively larger | Comparatively smaller |
| Configuration | spacer | spacer | spacer |
| Medicament | Pre-medication for | Therapeutic oncology | Post medication for |
| Role | allergic reaction | monoclonal antibody | allergic reaction |
| | prophlyaxis | | prophlyaxis |
| Medicament | Diphenhydramine | Oncomab | Dexamethasone |
| | | (Prototypical oncology | |
| | | mAb) | |
| Volume | 1 mL | 40 mL | 1.5 mL |
| Viscosity | 1 cP | 6.5 cP | 1 cP |
| Infusion Rate | Rapid (over 30-60 | Slow (over 60 | Rapid (over 30-60 |
| | seconds) | minutes) | seconds) |
| Depot Size | Very Small, largely | Large to Very Large, | Very Small, largely |
| | independent of | depending on | independent of |
| | physiology | physiology | physiology |
| Depot Reduction | Rapid (due to fast | Slow (due to slow | Rapid (due to fast |
| Rate vs. Time | absorption, small | absorption, large | absorption, small |
| Post-Infusion | volume, low viscosity) | volume, higher | volume, low viscosity) |
| | | viscosity) | |
| Separation from | Comparatively Small | Comparatively Large | Comparatively Small |
| Other Sites | | | |

In the example as shown in Table 1, the user may use the needle assembly connected to a medicament delivery device comprising these three medicaments and configured to deliver these three medicaments in a predetermined sequence. In this example, the spacing can be predetermined to avoid the potential skin wheal (depot) being overlapped in view of the delivery of Oncomab. Alternatively, the needle spacer arrangement may comprise at least three needle spacer elements or a needle guide with at least three guide surfaces, the three needle spacer elements or the three guide surfaces are configured to guide the placement of the three needles connected to these three medicaments respectively; as a result, the needle spacer elements or the guide surfaces for Diphenhydramine and Dexamethasone can be placed adjacent to one another, and the needle spacer element or the guide surface for Oncomab can be placed far away from the other two needle spacer elements or the guide surfaces. For example, the needle spacer elements or the guide surfaces for Diphenhydramine and Dexamethasone can be arranged to be placed on one abdomen side related to the umbilicus and the needle spacer element or the guide surface for Oncomab can be arranged to be placed on the other abdomen side related to the umbilicus.

It should be noted that if the user only needs to delivery Diphenhydramine and Dexamethasone, the user does not need the needle spacer arrangement, as these two medicaments are less likely to cause the skin wheal even if they are injected into sites that are adjacent to one another. In other words, in this example, the injection assembly as disclosed herein is designed for the delivery of Oncomab.

The size of the spacing is predetermined, based on at least one of the viscosities of first and the second predefined medicament, physiologic statistic data, the predefined administration rate of the first and the second predefined medicament, and the amount of the first and the second predefined medicament.

With predefined medicament administration is meant that a predefined type of drug/medicament is to be administered at a predefined rate, for a predefined amount of time. The target patient group is also predefined. This generally means that the expected size of a skin wheal is relatively well-defined especially if the skin wheal size variation based on the absorption capability of various users has been considered.

In one example, the injection assembly 1-1 comprises the needle spacer arrangement 5-1 which provides a spacing between the first needle 3a and the second needle 3b.

In the present example, the needle spacer arrangement 5-1 comprises a first needle spacer element 7a and a second needle spacer element 7b, which are physically separate objects.

The first needle 3a is mounted to the first needle spacer element 7a.

In a preferred example, the first needle is connected to a first counter needle mount 30 configured to be mounted to a first needle mount of the first needle spacer element. In one example, the first needle mount and the first counter needle mount 30 formed a mechanical fastener, e.g., a hook-and-loop fastener; alternatively, or additionally, the first needle mount and/or the first counter needle mount is adhesive.

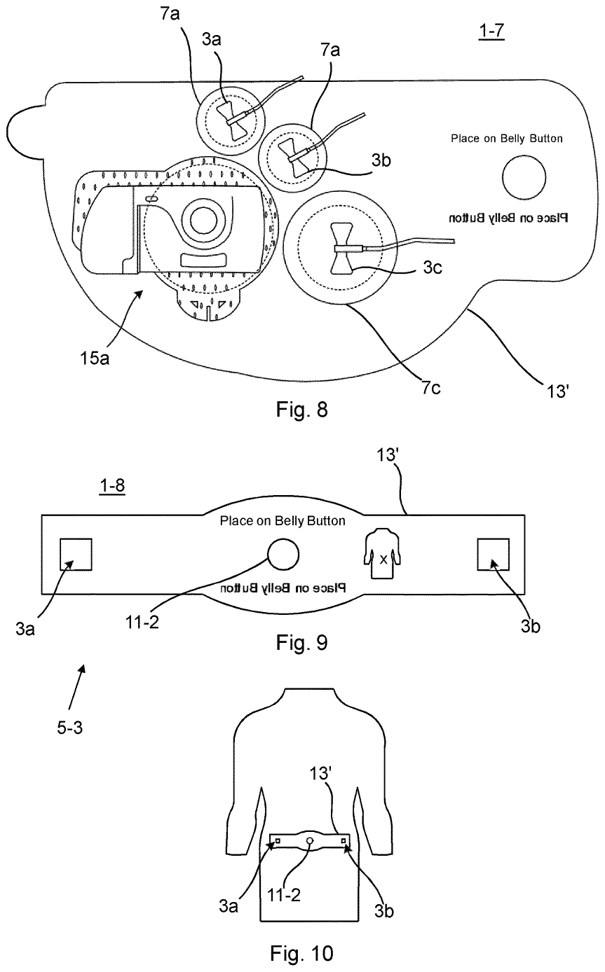

The first needle spacer element 7a is configured to be removably and operably attached to the skin of a user. The first needle spacer element 7a may for example comprise a layer of adhesive on its skin side, for attaching the first needle spacer element 7a to the skin, as shown in FIGS. 1A-4B. Alternatively, the first needle spacer element is attached to a needle guide (will be explained in detail later) configured to be removably attached to the skin of the user, as shown in FIGS. 7-8.

The second needle 3b is mounted to the second needle spacer element 7b. In a preferred example, the second needle is connected to a second counter needle mount configured to be mounted to a second needle mount of the second needle spacer element. In one example, the second needle mount and the second counter needle mount formed a mechanical fastener, e.g., a hook-and-loop fastener; alternatively, or additionally, the second needle mount and/or the second counter needle mount is adhesive.

The second needle spacer element 7b is configured to be removably and operably attached to the skin of a user. The second needle spacer element 7b may for example comprise a layer of adhesive on its skin side, for attaching the second needle spacer element 7b to the skin. Alternatively, the second needle spacer element is attached to a needle guide (will be explained in detail later) configured to be removably attached to the skin of the user.

In one example where the first needle comprises a first soft cannula and a second needle comprises a second soft cannula, the first needle spacer element 7a comprises a first needle inserter comprising a needle configured to insert the first soft cannula into user's body; and the second needle spacer element 7a comprises a second needle inserter comprising a needle configured to insert the second soft cannula into user's body. The first needle inserter is configured to retract the first needle once the first soft cannula is placed under the skin of the user; and the second needle inserter is configured to retract the second needle once the second soft cannula is placed under the skin of the user.

The first needle spacer element 7a may be dimensioned to provide spacing around the first needle 3a, larger than a skin wheal 9a that during the first predefined medicament administration is formed subcutaneously radially around the injection site, i.e., around the first needle 3a. The first spacer element 7a may thus have an outer perimeter 8a that is properly distanced from the first needle 3a.

The second needle spacer element 7b may be dimensioned to provide spacing around the second needle 3b, larger than a skin wheal 9b that during the second predefined medicament administration is formed subcutaneously radially around the injection site, i.e., around the second needle 3b. The second spacer element 7b may thus have an outer perimeter 8b that is properly distanced from the second needle 3b. Alternatively, or additionally, the first needle spacer element 7a and the second needle spacer element 7b may be dimensioned to provide such spacing around the first needle 3a and the second needle 3b, respectively, that they, if the needle spacer elements 7a, 7b with their respective two needles 3a, 3b are placed adjacent to each other, prevent mixing of medicament administered by means of the first needle 3a and the second needle 3b.

For example, the first needle spacer element has a first dimension, and the second needle spacer element has a second dimension. The first needle spacer element comprises a portion attached to the first needle and the second needle spacer element comprises a portion attached to the second needle. The first dimension and the second dimension are arranged such that when the first needle spacer element is adjacent to the second needle spacer element, the first needle and the second needle are spaced apart from one another with such a distance that the medicament administered by the first needle and the second needle are prevented from mixing subcutaneously and/or is larger than a diameter of an expected first skin wheal formed subcutaneously as a result of the first predefined medicament administration and a diameter of an expected second skin wheal formed subcutaneously as a result of the second predefined medicament administration.

In one example, the first needle spacer element and the second needle spacer element are flexible sheets. Additionally, the first needle spacer element and the second needle spacer element are flat sheets.

FIG. 1B shows when the first needle 3a and the second needle 3b have been positioned at the injection site for medicament administration. The first needle spacer element 7a and the second needle spacer element 7b are preferably arranged non-overlappingly. For example, the first spacer element 7a and the second spacer element 7b may be magnetic, arranged to repel each other. According to another example, the outer perimeters of the spacer elements 7a and 7b may be essentially perpendicular to a surface of a side that is arranged to face the user's body, and the spacer elements 7a, 7b may at the outer perimeter may have a relatively large thickness, such that overlapping placement of the spacer elements 7a and 7b would result in that the spacer element that is placed on top of a portion of the other spacer element loses touch with the user's body.

Alternatively, or additionally, the needle spacer elements can be placed on top of the needle or enclose the needle, as shown in FIGS. 33-36B. In this example, the needle may not be attached to the needle spacer element 7a′″. In this example, the user can place the needle to the skin before attaching the needle spacer element to the skin. Preferably, in this example, the needle spacer element comprises a cover 70′″ and a skin marker 71′″. The cover 70′″ is removable to the skin marker 71′″. For example, the skin marker 71′″ can be adhesive; in this example, the cover 70′″ can be glued to the skin marker with an adhesive being weaker than the adhesive between the skin marker 71′″ and the user's skin. Preferably, the cover 70′″ is transparent. The cover 70′″ is configured to avoid unintentionally disruption of the connection between the needle and the tube during use. In another example, the needle spacer element doesn't comprise the cover 70′″ as shown in FIGS. 36A-36B. In this example, the skin marker 71′″ is configured to enclose an area for the first needle to be placed onto such that when the second needle is placed outside of such area, the potential medicament mixing subcutaneously and/or overlapping of a skin wheat from the medicament delivered through the second needle can be avoid.

Preferably, a cut-out 72′″ is arranged to the skin marker 71′″ such that the tube 2 connected to the needle can extend through the cut-out 72′″, as shown in FIG. 34. In this example, the user can remove cover 70′″ after medicament delivery operation and remove the needle 3a. In this example, the skin marker 71′″ is remained on the skin of the user, as shown in FIG. 35. Thus, the user can get an indication that a following injection should not be carried out within the area being enclosed by the skin marker 71′″ of the needle spacer element 7a′″. Thus, for certain medicaments with low body absorption rate, a potential risk of the skin wheat or subcutaneously medicaments mixed under the user's skin from a following injection can be avoid. Furthermore, the remained skin marker 71″ also provides an indication to the user to avoid repeat injections on the same injection site. Furthermore, instead of being used together with the skin marker 71′″ that is configured to enclose a medicament delivery site, as shown in FIG. 38A, the cover 70″″ can be used together with the needle spacer element 7a that is configured to be placed between the skin and the needle, as shown in FIG. 1A. Preferably, the cover 70″″ can be used to remove the needle 3a, as shown in FIG. 38B.

Furthermore, optionally, at least one of the first and the second needle spacer element 7a, 7b comprises a tube retention fastener, as shown in FIG. 37. For example, the retention fastener can be made of material has high Coefficient of friction, e.g., rubber, such that the tube 2 is held by the tube retention fastener via friction. Alternatively, or additionally, the retention fastener is configured to engage with a counter tube retention fastener of the tube 2. For example, the retention fastener and the counter tube retention fastener can form a thread fastener or a bayonet fastener. In this example, the connection between the needle and the tube can be further secured.

FIG. 2 shows another example of an injection assembly. The injection assembly 1-2 comprises a needle spacer arrangement 5-2, which comprises the first needle spacer element 7a, the second needle spacer element 7b, and additionally a needle guide 13. The needle guide 13 is a physically separate object relative to the first needle spacer element 7a and the second needle spacer element 7b.

According to the present example, the injection assembly 1-2 also comprises a third needle spacer element 7c and a third needle 3c mounted to the third needle spacer element 7c. The third needle spacer element 7c is according to the example identical in size to the first needle spacer element 7a.

The needle guide 13 has an outer contour 11-1 which forms a first guide surface 11-1a and a second guide surface 11-1b. The first guide surface 11-1a is adapted to an outer contour of the first needle spacer element 7a for positioning the first needle spacer element 7a relative to the needle guide 11-1. The second guide surface 11-1b is adapted to an outer contour of the second needle spacer element 7b for positioning the second needle spacer element 7b relative to the needle guide 13.

In the example in FIG. 2, the outer contour 11-1 also forms a third guide surface 11-1c adapted to an outer contour of the third needle spacer element 7c for positioning the third needle spacer element 7c relative to the needle guide 13.

According to the example, the first guide surface 11-1a is configured to mate with the first needle spacer element 7a. The second guide surface 11-1b is configured to mate with the second needle spacer element 7b. The third guide surface 11-1c is configured to mate with the third needle spacer element 7c.

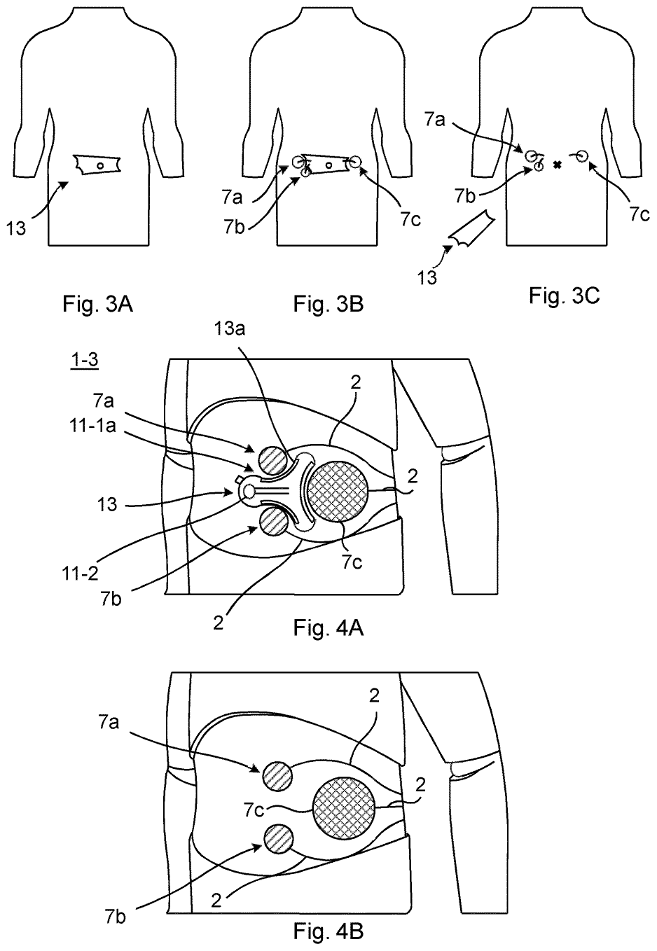

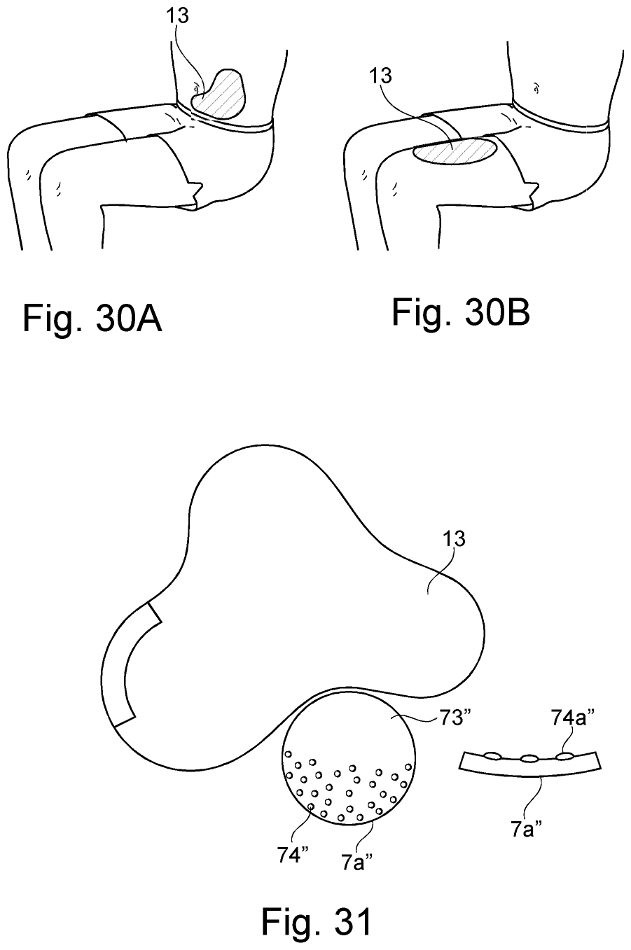

The needle guide 13 according to the example comprises a location through-opening 11-2 defining an anatomical reference point, as shown in FIGS. 3A-16, and FIG. 22. In the present example the needle guide 13 is shaped to be attached to the abdomen, and the anatomical reference point is the umbilicus. The shape of the needle guide may for example suggest anatomic placement on the abdomen and will in this case clearly not fit a thigh or back-of-arm injection site, as shown in FIGS. 30A-30B. Features may also be suggestive of (i.e., encourage) a desired anatomic site, or alternatively, may be contoured to discourage an undesired anatomic site.

In one example, the needle guide 13 has an asymmetrical shape with respect to an axis A that divides the needle guide 13 in two through the location through-opening 11-2. The first and third needle spacer element 7a and 7c are arranged at a respective side of the location through-opening 11-2. The second needle spacer element 7b, which according to the example is smaller than the first and third needle spacer element 7a and 7c is arranged adjacent to the first needle spacer element 7a mated with the second guide surface 11-1b.

The first needle 3a and the third needle 3c may be used for a small molecule medicament (first medicament). Their location relative to each other ensure that large volumes may be delivered simultaneously with maximum tolerability. The second needle 3b may be used for a second medicament.

The needle guide 13 may be provided with an adhesive so that it can be removably attached to the user's skin during the entire medicament administration process. Alternatively, the needle guide 13 may be removed before the medicament administration process commences, as shown in FIGS. 3A-3C, where the needle guide 13 is first placed on the abdomen, followed by positioning of the first, second, and third needle spacer elements 7a-7c using the needle guide 13, wherein the needle guide 13 is removed.

According to one example, the needle guide may be held by the user and pushed on the skin. According to one example, the needle guide may be provided with a wearable feature, e.g., a shoulder/neck strap, a waist/leg belt, a garment, a belt clip, or a leg sleeve for attaching the needle guide to the user's body.

The needle guide may be a sheet, such as a flat or planar sheet. The needle guide may be flexible. The needle guide may be formed as a single component or may be formed of several parts. The needle guide may be according to one example comprise a handle for allowing a user to hold the needle guide fixed relative to the user's body. According to one example, the needle guide may have different thickness in different areas, to enable control of different injection depths.

FIG. 4A shows another example of an injection assembly. The injection assembly 1-3 comprises a needle spacer arrangement 5-2, which comprises the first needle spacer element 7a, the second needle spacer element 7b, and a needle guide 13 which is physically separate from the first needle spacer element 7a and the second needle spacer element 7b.

Each needle is connected to a medicament delivery device by means of a respective tube 2.

In this example, the needle guide 13 has an outer contour 13 which forms a first guide surface 11-1a and a second guide surface 11-1a. The first guide surface 11-1a is adapted to an outer contour of the first needle spacer element 7a for positioning the first needle spacer element 7a relative to the needle guide 11-1. The second guide surface 11-1b is adapted to an outer contour of the second needle spacer element 7b for positioning the second needle spacer element 7b relative to the needle guide 13.

In the example in FIG. 4A, the outer contour 13 also forms a third guide surface 11-1c adapted to an outer contour of the third needle spacer element 7c for positioning the third needle spacer element 7c relative to the needle guide 11-1.

According to the example, the first guide surface 11-1a is noncongruent with the first needle spacer element 7a. The second guide surface 11-1b is noncongruent with the second needle spacer element 7b. The third guide surface 11-1c is noncongruent with the third needle spacer element 7c.

In the present example, each of the first, second and third needle spacer elements 7a-7c has a colour that matches the corresponding first, second and third guide surface 11-1a to 11-1c. For example, the first guide surface 11-1a may be marked with a first colour 13a and the first needle spacer element 7a may also be marked with the first colour. The second guide surface 11-1b may be marked with the first colour and likewise the second needle spacer element 7b may be marked with the first colour. The third guide surface 11-1b may be marked with a second colour, different from the first colour, and the third needle spacer element 7c may be marked with the second colour. The colour that is used may be based on the size of the needle spacer elements 7a-7c. For example, if two needle spacer elements have the same size, they may be provided with the same colour. Using colour matching may facilitate the positioning of the needle spacer elements 7a-7c when the surfaces are noncongruent as in this example. However, it is envisaged that colour matching could be provided also in e.g., the example in FIG. 2. The colours may for example be selected such that they are distinguishable by those users with poor eyesight, certain colour-blindness, or colour vision deficiency. Alternatively, or additionally, a texture guide can be provided, as shown in FIG. 31. For example, the first guide surface and the second guide surface can be formed as smooth surfaces; and a portion 73″ of the first needle spacer element 7a″ can be formed with a smooth surface, and the rest portions of the first needle spacer element 7a″ can be formed with a rough surface 74″, e.g., multiple protrusions 74a″ can be provided; similarly, a portion of the second needle spacer element can be formed with a smooth surface, and the rest portions of the second needle spacer element can be formed with a rough surface. In this example, the user can be guided to place the smooth surfaces 73″ of the needle spacer elements 7a″ adjacent to the smooth surface of the needle guide 13.

FIG. 4B shows the injection assembly 1-3 during medicament administration, when the needle guide 13 has been removed from the body of the user. Alternatively, the needle guide 13 may be maintained on the body of the user also during medicament administration.

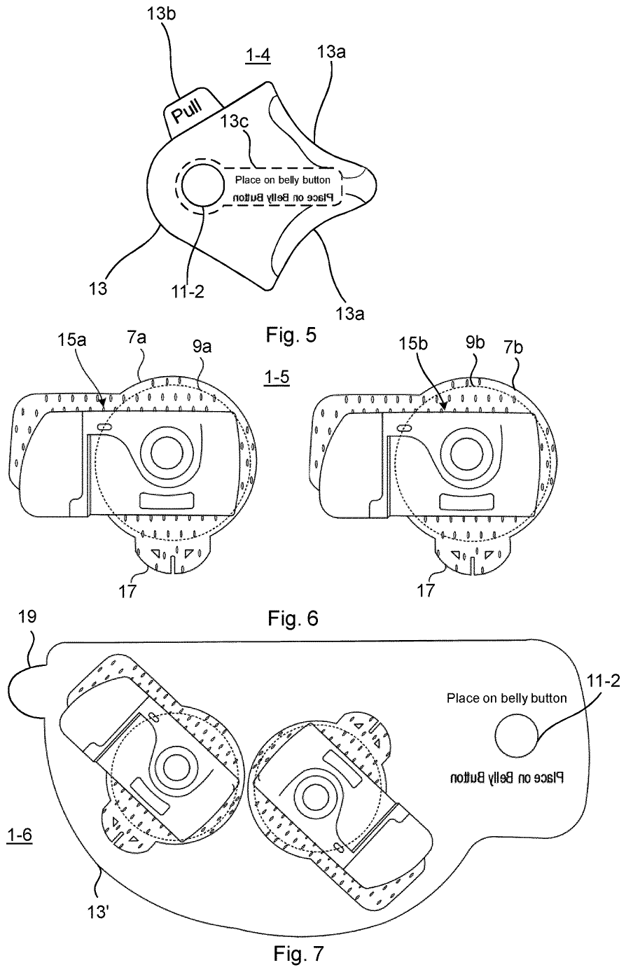

It is to be appreciated that the number of needle spacer elements/needles, and the size of the needle spacer elements may vary in different variations of the example in FIGS. 4A-4B. For example, as shown in FIG. 5, the injection assembly 1-4 (comprising the needle spacer arrangement being another example of the needle spacer arrangement 5-2) may have a needle guide 13 configured to accommodate only two needle spacer elements of the same size.

In the example in FIG. 5, the needle guide 13 comprises a tab 13b which facilitates pulling off the needle guide 13 from the user's skin. Moreover, the needle guide 13 has an outer surface, facing away from the user's skin during use, provided with an instructional text and reversed instructional text 13c describing an anatomical site of the anatomical reference point. The instructional text may for example state “place on belly button” both right reading and reversed. Such an instructional text may be provided on any of the examples of the needle guide disclosed herein where text is present, for example, on tab 13b.

FIG. 6 shows another example of an injection assembly. The injection assembly 1-5 (comprising the needle spacer arrangement being another example of the needle spacer arrangement 5-1) comprises two medicament delivery devices 15a and 15b. Each medicament delivery device 15a and 15b may be an on-body injector comprising a medicament container and a pump configured to pump the medicament from the medicament container. The medicament delivery devices 15a and 15b may alternatively, according to some examples be needle insertion devices or cannula insertion devices connected to a respective tubes 2a, 2b.

Each medicament delivery device 15a, 15b may be arranged with a different medicament, or they may contain the same medicament.

Each medicament delivery device 15a, 15b comprises a respective needle (not shown). Each medicament delivery device 15a and 15b is mounted to a respective needle spacer element 7a and 7b. No tubes are required as the medicament is contained locally in the medicament delivery devices 15a, 15b. The size of the expected skin wheal 9a, 9b is shown by a dashed circle. As long as the first and the second needle spacer elements 7a and 7b are arranged non-overlappingly, there will be a distance between the skin wheals 9a and 9b.

Each of the first and the second needle spacer element 7a and 7b may comprise a tab 17 with an adhesive that reaches radially beyond the expected size of the skin wheal 9a.

FIG. 7 shows another example of an injection assembly. The injection assembly 1-6 (comprising the needle spacer arrangement being another example of the needle spacer arrangement 5-2) comprises two or more medicament delivery device 15a, 15b, each mounted to a respective one of the first needle spacer element 7a and the second needle spacer element 7b, as depicted in FIG. 6. Further, the needle spacer arrangement of the injection assembly 1-6 also comprises a needle guide 13′.

The needle guide 13′ comprises a first opening configured to receive the first needle spacer element 7a and a second opening configured to receive the second needle spacer element 7b. The needle guide 13′ could optionally comprise more than two openings for the two needle spacer elements 7a, 7b such that the user may vary the position of needle spacer elements 7a, 7b. In this example, the first guide surface of the needle guide is defined by the contour of the first opening; and the second guide surface of the needle guide is defined by the second opening.

The needle guide 13′ also comprises the location through-opening 11-2 defining an anatomical reference point. In this example, the anatomic reference point is the umbilicus.

The needle guide 13′ may comprise a tab 19 arranged such that a user may rotate the needle guide 13′ about the anatomical reference point holding the tab 19. This way, an appropriate orientation/location on the skin may be found relative to the anatomical reference point.

According to one example, another needle spacer arrangement 5-3 is provided. In this example, the needle spacer arrangement comprises the needle guide 13′ and does not comprise the needle spacer element(s). In this example, one or both of the medicament delivery devices 15a and 15b may be provided without a needle spacer element and installed directly into or onto the needle guide 13′ instead.

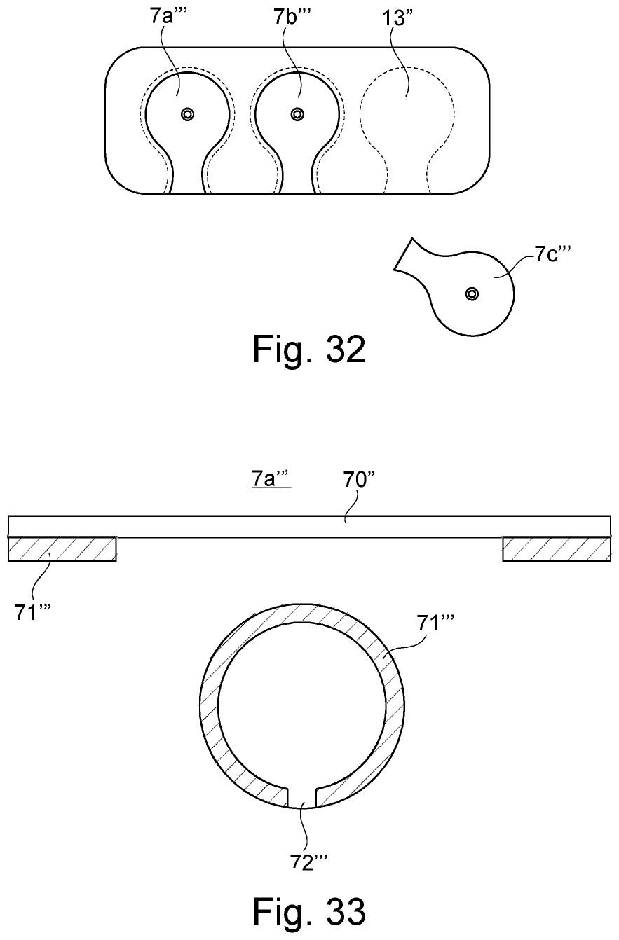

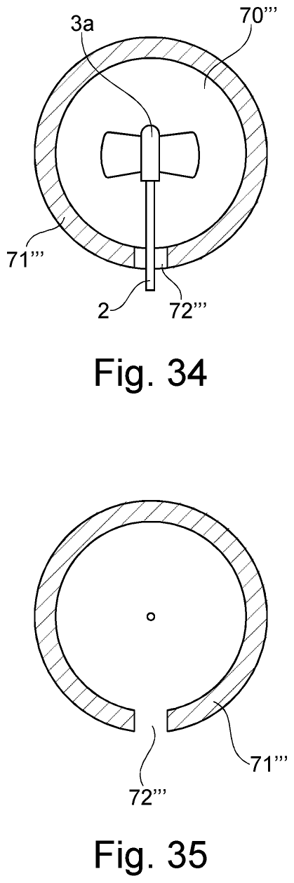

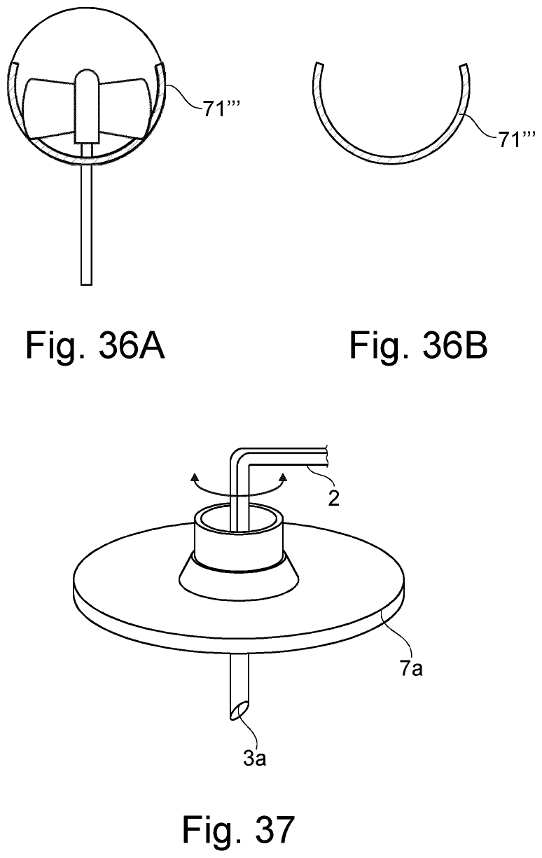

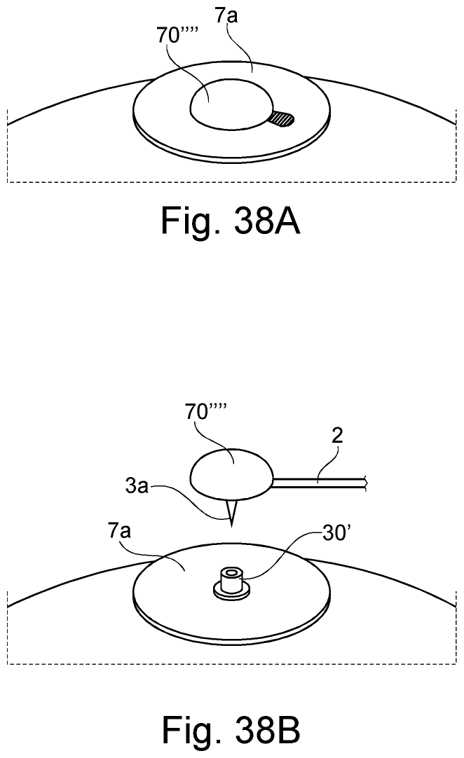

The example of an injection assembly 1-7 (comprising the needle spacer arrangement being another example of the needle spacer arrangement 5-2) shown in FIG. 8 uses needles 3a-3c with corresponding needle spacer arrangements 7a-7c connected by tubes to remote medicament delivery device(s), and a medicament delivery device 15a of the type described in FIGS. 6-7, mounted to a needle spacer element, in combination with a needle guide 13′ similar to the type shown in FIG. 7. Each needle spacer arrangement 7a-7c is arranged in a respective opening in the needle guide 13′. This configuration may be advantageous when the medicament delivery device 15a contains an emergency medicament that may be contingently administered in response to an adverse, unexpected, or unwanted event in response to administration of one or more medicaments through the needles 3a-3c, such as a systemic reaction. Alternatively, the needle guide may comprise an extra opening to highlight an injection site for receiving an emergency injection. In this example, the extra opening will only receive a needle in an emergency event that the user can inject on the site that is enclosed by the extra opening. Alternatively, or additionally, in one example, the needle guide 13″ comprises the needle spacer elements 7a′″, 7b′″, 7c′″ as shown in FIG. 32. In this example, the needle spacer elements 7a′″, 7b′″, 7c′″ are removable to the needle guide 13″, for example, the needle spacer elements 7a′″, 7b′″, 7c′″ are connected to the needle guide 13″ with perforated connections. Thus, more design flexibility can be provided. For example, the user can remove some needles together with the needle spacer elements and keep some needles in places.

According to one example, one or more medicament delivery devices 15a, 15b may be provided without a needle spacer element and installed directly into or onto the needle guide 13′ instead, as mentioned above as comprising the needle spacer arrangement being another example of the needle spacer arrangement 5-3, and the same may apply to the needles too. Furthermore, another needle spacer arrangement can be provided. In this example, the needle spacer arrangement comprises the needle guide 13′ comprising multiple openings. Some openings of the needle guide 13′ are configured to receive multiple needles respectively without needle spacer element; and some openings of the needle guide 13′ are configured to receive multiple needle spacer elements respectively. Additionally, the needle guide 13′ comprises the outer contour with one or more guide surface 11-1a, 11-1b, 11-1c configured to guide the one or more needle spacer elements to be placed relative to the needle guide respectively as mentioned above.

FIG. 9 shows another example of an injection assembly. The injection assembly 1-8 comprises a needle spacer arrangement 5-3 that includes a needle guide 13′ and does not include the needle spacer element. The needle guide 13′ may be provided with an adhesive to removably attach the needle guide 13′ to the user's skin.

The needle guide 13′ also comprises the location through-opening 11-2 defining an anatomical reference point. In this example, the anatomic reference point is the umbilicus.

In this example, the needle guard 13′ comprises the first opening and the second opening. The injection assembly 1-8 further comprises a first needle 3a and a second needle 3b, which are attached to the needle guide 13′. The first needle 3a extends through the first opening, and the second needle 3b extends through the second opening.

The needle guide 13′ is generally I-shaped. The first needle 3a is arranged at a first end of the I and the second needle 3b is arranged at a second end of the I, opposite to the first end.

In this example, the needle guide 13′ provides the spacing between the first needle 3a and the second needle 3b.

The needle guide 13′ is in this example symmetrical with respect to the location through-opening 11-2.

FIG. 10 depicts when the injection assembly 1-8 is attached to the user's skin.

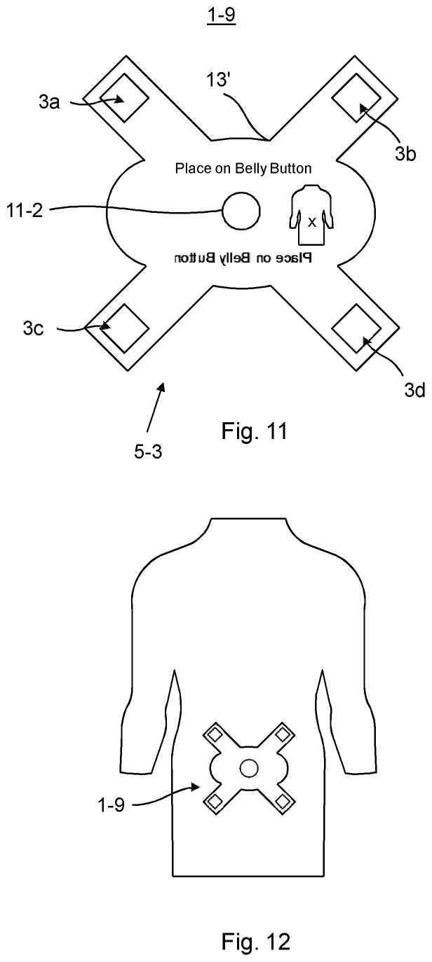

FIG. 11 shows yet another example of an injection assembly. The injection assembly 1-9 comprises a needle spacer arrangement 5-3 that includes a needle guide 13′ and does not include the needle spacer element. The needle guide 13′ may be provided with an adhesive to removably attach the needle guide 13′ to the user's skin. Other alternative attachment possibilities are also envisaged, such as straps, as previously described.

The needle guide 13′ also comprises the location through-opening 11-2 defining an anatomical reference point. In this example, the anatomic reference point is the umbilicus.

Further, the injection assembly 1-9 comprises a needle guide 13′ having a first opening, a second opening, a third opening and a fourth opening. The injection assembly comprises a first needle 3a configured to extend through the first opening, a second needle 3b configured to extend through the second opening, a third needle 3c configured to extend through the third opening, and a fourth needle 3d configured to extend through the fourth opening, which are attached to the needle guide 13′.

The needle guide 13′ is generally X-shaped. The needles 7a-7d are arranged in respective arm or leg of the X. Specifically, the needles 7a-7d may be arranged in an end of a respective arm of or leg of the X.

In this example, the needle guide 13′ provides the spacing between the needles 3a-3d.

The needle guide 13′ could alternatively be generally Y-shaped. In this case, for example, three needles may be fitted to the needle guide 13′, one in each leg or arm of the Y.

FIG. 12 illustrates when the injection assembly 1-9 has been attached to the user's skin, centred on the umbilicus.

In variations of the examples in FIGS. 9 and 11, the needle guide 13′ could be removed after the needles have been positioned properly on the user's skin, before the medicament administration is commenced. The needles are in this case left removably attached to the skin of the user. Alternatively, the needle guide 13′ may be maintained on the user's skin during medicament administration.

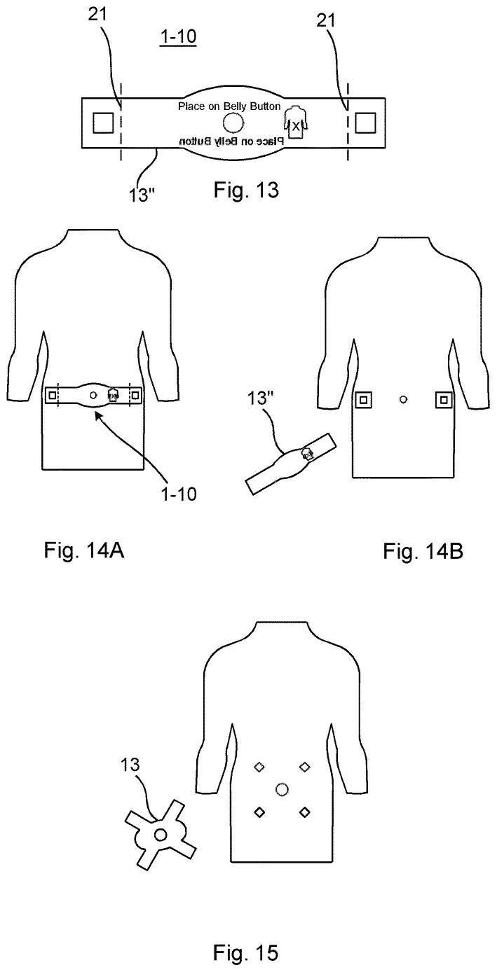

According to one example, shown in FIG. 13 and FIGS. 14A-14B, the injection assembly 1-10 may comprise a needle guide 13″ provided with perforated sections 21 and/or pull tabs to remove a majority of the needle guide 13″ once it has been used to position the needles 3a, 3b on the user's skin, before medicament administration is commenced. FIG. 15 similarly shows the removal of the needle guide 13″ prior to medicament administration when it is generally X-shaped as in the example in FIGS. 11-12.

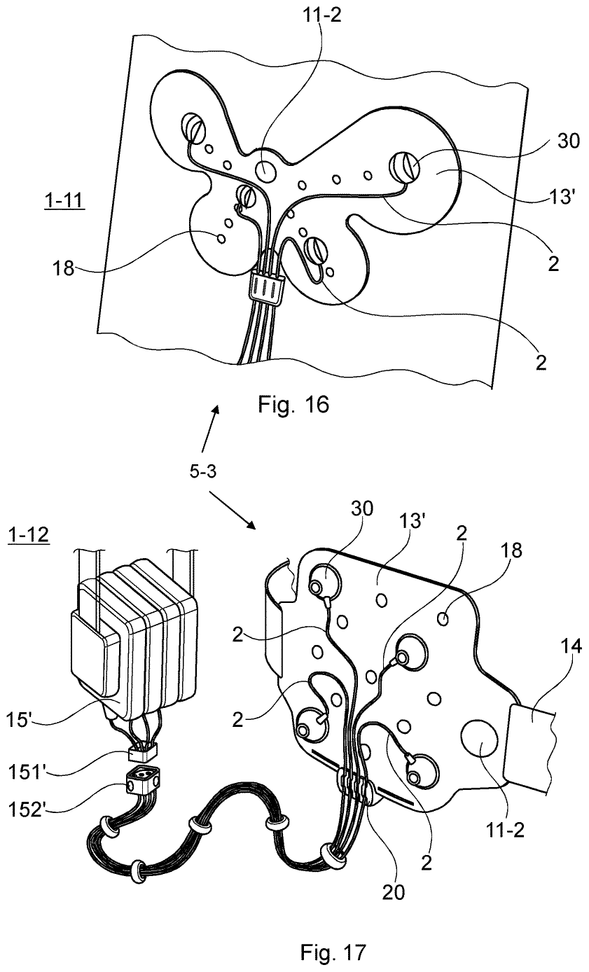

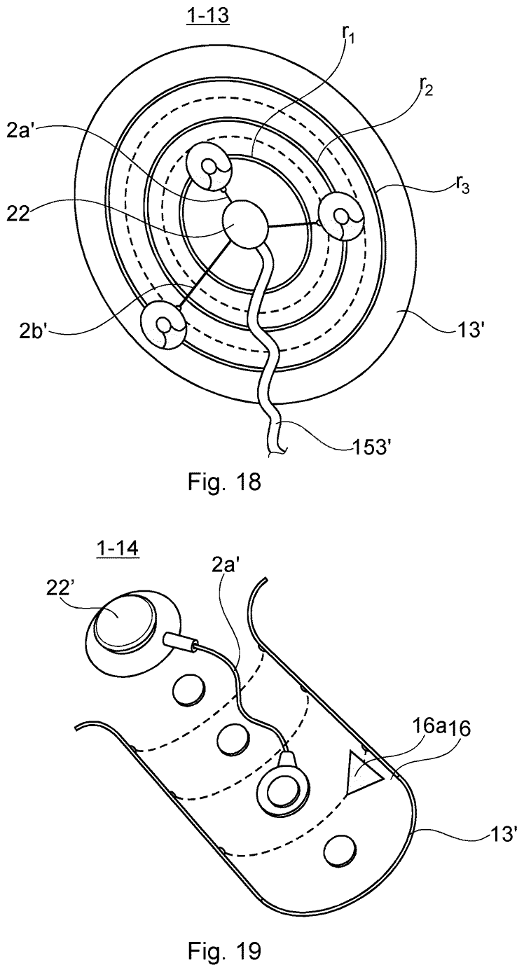

Furthermore, according to another example, the needle guide 13′ of the needle spacer arrangement 5-3 of the injection assembly 1-11 and 1-12 comprises multiple openings 18, as shown in FIGS. 16-17. In this example, the needle guide 13′ can be a universal needle guide that can guide the location of the needle for different injections/infusion. In other words, as different medicament has different absorption rate in the human body so that the size of the expected skin wheal might be different regarding different medicament delivery. As a result, when the user receives different medicament, the distance between needles might be different so that the medicament mixing subcutaneously or skin wheals overlapping can be effectively avoided. In one example, the user might use different needle guides for different medicament delivery. Alternatively, the user can use the universal needle guide 13′ as shown in FIGS. 16-17. In this example, the multiple openings are designed for being passed through by multiple needles respectively. As a result, the distance between the openings provides a spacing between needles. In one preferred example, the needle guide comprises multiple identifying features, e.g., colour codes, marks, and/or shapes, for different groups of the multiple openings to differentiate the different groups of the multiple openings from other groups of the different groups of the multiple openings. For example, one group of the multiple openings comprises four openings located at a predetermined distance from one another. The distance is predetermined to avoid the medicament mixing subcutaneously or skin wheals overlapping when delivering a specific type of medicament. In this example, the same colour can be used around these four openings, so that the user can be aware that this group of the multiple openings is configured to receive needles when the specific type of medicament delivery is taking place.

Similarly, the needle guide having multiple openings and identifying features as disclosed in this embodiment can also be used with the needle spacer elements as mentioned above. For example, in one example where the needle spacer arrangement comprises the needle guide having the first guide surface and the second guide surface; the first needle spacer element, and the second needle spacer element; and the first and the second needle spacer element are configured to be placed adjacent to the first and the second guide surface respectively, the openings 13a, 13b′, 13c′ of the needle guide 13 may be shaped differently for different needle spacer elements 7a′, 7b′, 7c′ that are configured to be used with different medicaments, as shown in FIG. 29.

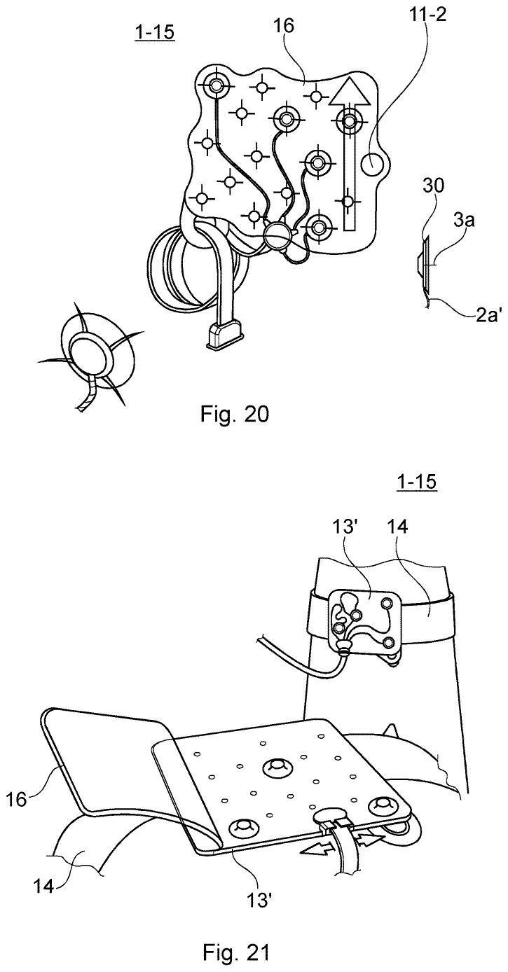

As mentioned above, the medicament delivery device may be remote to the medicament delivery sites on the user's skin, and the medicament delivery device is connected to needles via tubes 2. Thus, to avoid one or more tubes from tangling, in any example comprising tubes and a needle guide, the injection assembly may comprise an optional tubing management hub configured to retain the tubes to prevent kinking or inadvertent dislodging. The needles and the tubing management hub may according to one example be formed as a single integrally moulded component, for example, made of an elastomeric material such as silicone rubber. In another preferred example, the injection assembly comprises tubes 2 fluidly connected to the medicament delivery device 15′. In another preferred example, the injection assembly comprises tubing management hub 20, configured to retain the tubes. In another example, the tubing management hub 20 that can be attached to the needle guide 13′ via a clip or an adhesive attachment, as shown in FIG. 17. In one example, the medicament delivery device 15′ might comprise multiple medicament containers connected to multiple tubes respectively; or one medicament container connected to multiple tubes. In another example, the medicament delivery device 15′ comprises a tubing connecting hub 151′ configured to be attached to a counter tubing connecting hub 152′. In one example, the needle guide 13′ comprises multiple needle mounts. In one example, multiple needle mounts can be arranged around one opening of the multiple openings; alternatively, one needle mount is arranged adjacent to one opening of the multiple openings. In this example, the needles 3 are connected to counter needle mounts configured to be mounted to the needle guide by being attached to the needle mounts. For example, the needle guide comprises a first needle mount adjacent to the first opening, and a second needle mount adjacent to the second opening. The first needle 3a and the second needle 3b are configured to be releasably mounted to the first and the second needle mounts of the needle guide. In this example, the user doesn't need to attach the needle to the skin via the adhesive material. Therefore, the user experience can be improved. In one example where the needle guide comprises multiple openings for receiving multiple needles respectively, the needle guide comprises multiple needle mounts adjacent to the multiple openings respectively. In this example, at least one needle mount of the multiple needle mounts comprises an identifying feature to differentiate the at least one needle mount from the rest of the multiple needle mounts. In one example, the needles are connected to counter needle mounts 30 configured to be attached to the needle mounts of the needle guide. In one example, the tubes are connected to the needles via the counter needle mounts 30, as shown in FIG. 20. In another example, as shown in FIGS. 27A-27B, the needle mount 130 of the needle guide 13′ can be a mechanical fastener, e.g., hook-and-loop fasteners, and the counter needle mount 30′ can be a counter mechanical fastener. In this example, the needle assembly comprises a right-angle needle 3a connected to a tube 2a. In this example, no adhesive is needed for the needle assembly, as the needles and the needle guide can be attached to the skin via the mechanical fasteners (for the needle) and the wearable feature (for the needle guide). Other mechanical fasteners can also be used in this example such as buttons. Alternatively, the needles can be attached to the needle guide via the adhesive instead of being attached onto the skin directly. Alternatively, some of the needle mounts and the corresponding counter needle mounts, e.g., the first needle mount and the first counter needle mount, can formed a hook-and-loop fastener; and some of the needle mounts and the corresponding counter needle mounts, e.g., the second needle mount and the second counter needle mount, can be adhesive.

It should be noted that in the examples as shown in FIGS. 7-8 where the first and the second needle are configured to be attached to the skin of the user via the first and the second needle spacer element and the needle guide, the counter needle mounts can be provided by the first and the second needle spacer element.

It should be noted that in the example where the first needle comprises the first soft cannula, and the second needle comprises the second soft cannula, the tube 2a, 2b′ as mentioned above can be a part of the first and the second soft cannula. In other words, instead of connecting to needles via tubes 2, the medicament delivery device is connected to the first and the second soft cannula.

The fastener between the needle mount and the counter needle mount mentioned in all examples above provides a safety mechanism to avoid the needles from being pulled when the user accidentally pulls the tubes. Thus, the user can be protected from injury by the needles.

In another example, as shown in FIGS. 18-19, the injection assembly 1-13, 1-14 comprises a distributing hub 22; 22′ connected to the tubes 2a′, 2b′ and the medicament delivery device. In this example, the distributing hub 22 is configured to be connected to a main tube 153′ extending from the medicament delivery device. The delivered medicament is configured to enter into each tube connected to a needle from the distributing hub 22. In this example, the distributing hub 22; 22′ is configured to be used with the needle spacer arrangement 5-2; 5-3 includes the needle guide 13; 13. In one example, the distributing hub 22 is attached to the needle guide 13′. Alternatively, the distributing hub is an integral part of the needle guide. In one example, the needle guide 13′ is circular, as shown in FIG. 18, in this example, the distributing hub 22 can be arranged at the centre of the needle guide 13′. In this example, the distance between needles can be set by the distance between the needle and the distributing hub. For example, the needle guide can be coated with three circles coaxially arranged relative to the distributing hub 22, as shown in FIG. 18. Each circle is distant from the distributing hub 22 with a predetermined distance r1, r2, r3. The user can be instructed to place the first needle on the second circle, and the second needle on the third circle for delivering a specific medicament. Alternatively, or additionally, the distance between needles can be determined by angles of the circle. Alternatively, the distributing hub 22′ can be placed on one side of the needle guide 13′ as shown in FIG. 19. Furthermore, in another example, the needle guide 13′ of the injection assembly 1-14, 1-15, 1-17 comprises a cover 16. The cover is configured to seal the openings of the needle guide to avoid any potential contamination before use and/or disruption during use. The cover is configured to be removed before placing the needles on the needle guide 13′ and/or to be placed on the needles and the openings after the needles are placed on in the openings respectively of the needle guide 13′. In one example, the cover is disposable. Alternatively, the cover 16 is reusable. In one example, as shown in FIGS. 20-21, 23, the cover 16 covers the needles during medicament delivery operation. In a preferred example, the cover 16 comprises a tab 16a configured to be gripped by the user to remove the cover 16. In this example, the cover will be removed for placing the needles, as shown in FIG. 21, 23, and reattached to the needle guide afterwards, as shown in FIG. 20. In one example, the cover is made of flexible material. Alternatively, or additionally, instead of arranging one cover to cover multiple openings of the needle guide 13′, multiple covers can be provided. In this example, one cover is used to cover one opening of the multiple openings 18 of the needle guide 13′.

In another example, the injection assembly 1-16 comprises multiple needle guides 13′. In one example, the needle guides can be partially overlapped with one another.

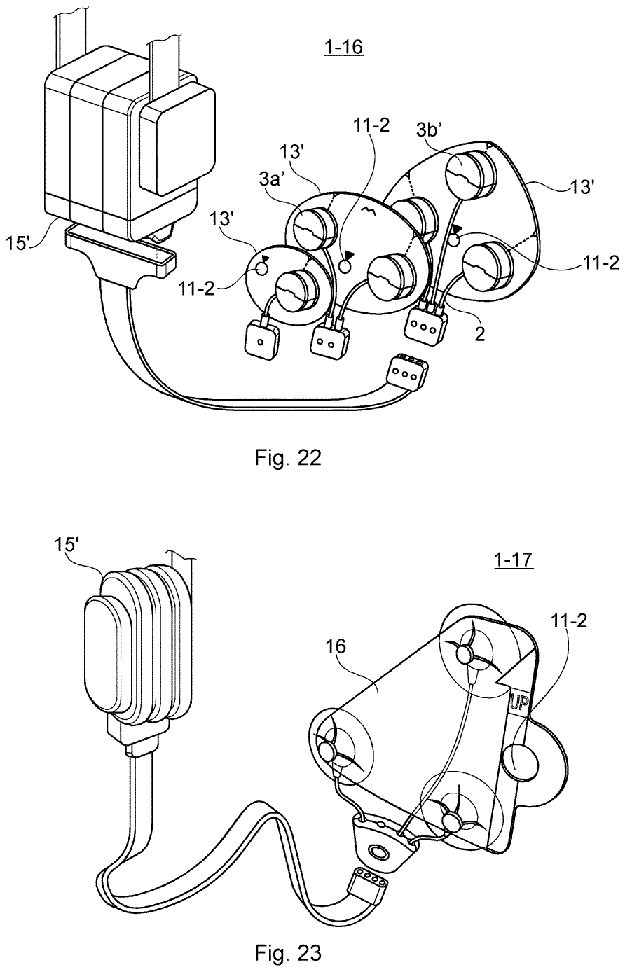

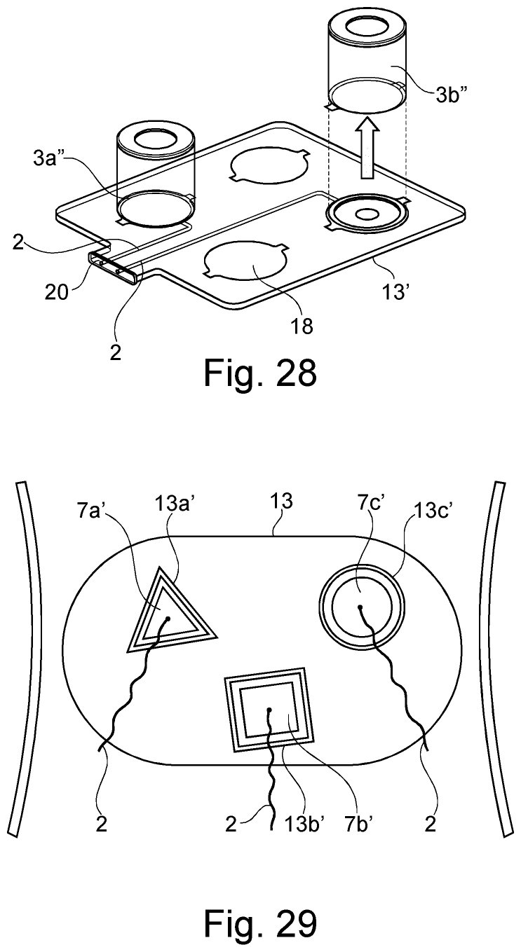

Optionally, the needle guide as disclosed in any example above comprises a through-opening 11-2 defining an anatomical reference point; and/or a user behaviour guide, e.g., colour codes, texture, marks, instructions . . . etc, to guide the user to place the needles on/adjacent to the needle guide. Furthermore, optionally, the needle guides as disclosed in any example above comprise a wearable element 14 for being worn by the user, so that the needle guide can be releasably attached to the skin of the user via the wearable element. In a preferred example, the wearable element can be a belt, a shoulder stripe, a neck stripe, a garment, a leg sleeve, or a belt clip. Instead of attaching the needle guide to the skin via an adhesive, the wearable element will not tear the skin during removal. Thus, the user experience can be improved. Furthermore, the needle guides as disclosed in any example above, instead of mounting needles on the needle guide, the needles and tubes can be an integral part of the needle guide. Furthermore, instead of the needles, one or more needle inserters 3a′, 3b′ can be attached to the needle guide 13′, as shown in FIG. 22. In this example, the needle inserter can be a part of the needle spacer element as mentioned above. In this example, the needle is held by the needle inserter 3a′, 3b′ and is configured to extend through the opening of the needle guide to penetrate the skin of the user. In another example, as shown in FIG. 28, the needle inserter 3a″, 3b″ comprises the needle. In this example, the tube 2 is arranged within the needle guide 13′ For example, the needle guide 13′ comprises a first body layer and a second body layer placed on top of the first body layer. The tube 2 is positioned between the first and the second body layers. In a one example, the needle comprises a through-hold opening in a direction transverse to the needle extending direction. In this example, the through-hold of the needle is configured to be lined up with the tube 2 when the needle penetrated through the skin of the user; alternatively, the tube 2 is fluidly connected to a soft cannula. In this example, the soft cannula extends between a needle opening and a skin opening. In this example, the needle of the inserter is configured to enter the soft cannula from the needle opening and insert the soft cannula together with the needle into user's body. In a preferred example, the needle is configured to be retracted after the soft cannula is positioned under the user's skin. In a preferred example, the needle is configured to be retracted to a position that the needle seals the needle opening of the soft cannula; in this example, the needle is served as an insertion needle. Furthermore, the needle guide can comprise a lock feature to lock the needle inserter to the needle guide. For example, the needle inserter comprises a round edge with one protrusion matching to a recess of the needle guide comprising a cut-out. In this example, the needle inserter can be placed on the needle guide by being lined up the protrusion with the cut-out. The user can then twist the needle inserter within the recess relative to the needle guide. As a result, the protrusion is offset from the cut-out such that the needle inserter cannot be removed from the needle guide.

It should be noted that in one example where the needle guide 13; 13′ is not configured to be removed during medicament delivery operation, the needle guide 13; 13′ may comprises the tube retention fastener as mentioned above. For example, the tube retention fastener can be arranged adjacent to the openings 18 of the needle guide 13′.

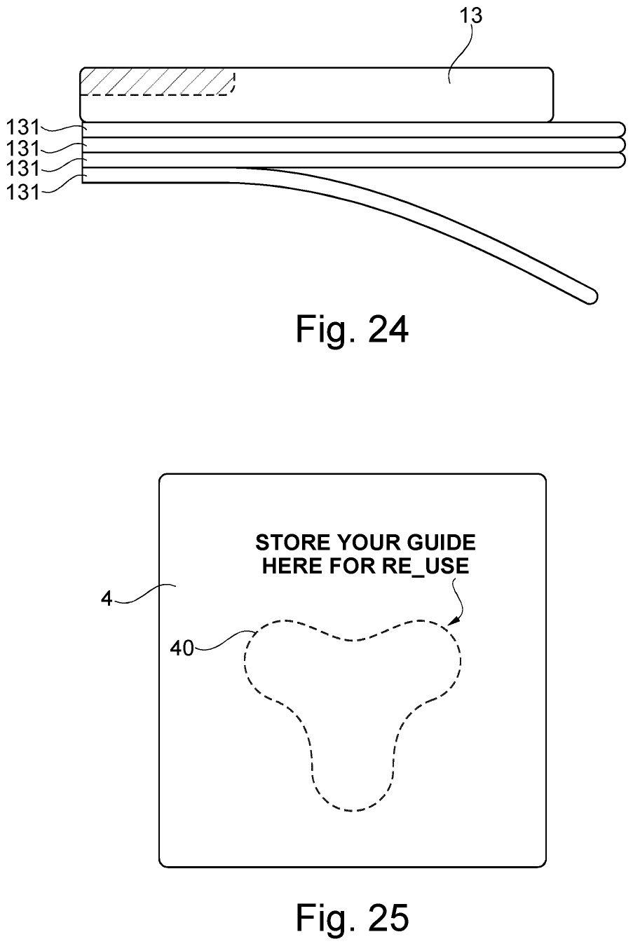

Furthermore, in one example where the needle guide 13; 13′ comprises the adhesive, the needle guide comprises an adhesive material, as the mentioned adhesive, arranged on the inner surface of the needle guide configured to be removably attached to the skin of the user. Preferably, the needle guide 13 comprises multiple layers 131 of adhesive materials arranged on the inner surface of the needle guide 13 configured to be removably attached to the skin of the user, as shown in FIG. 24. In this example, a layer of the multiple layers of adhesive materials is configured to be removably attached to the skin of the user, and the layer that is attached to the skin of the user is configured to be removed from the needle guide after use of the needle assembly.

Furthermore, a kit of the needle assembly can be provided. In one example the kit comprises a needle guide 13; 13′, and a needle guide storage 4. In this example, the inner surface of the needle guide 13; 13′ is configured to be attached to the needle guide storage 4. In one example where the needle guide comprises the adhesive, the adhesive may be adhered to this storage between uses. The needle guide storage 4 can be a card, a plate, or a box. The needle guide storage may be opaque or transparent. Additionally, the size and shape of the needle guide storage may be more easily stored (e.g., in a folder, propped against a shelf) and be more easily identified and selected from other infusion materials stored in less organized ways (e.g., multiple supply types mixed in a drawer or bin). In another example, the needle guide storage may contain branded colours and other medication indicia such as brand name or regimen overview. Additionally, the storage card contains personalization elements such as a place to write the patient's name.

Optionally, another surface, e.g., the reverse surface, of the storage contains instructional text on proper use and storage of the needle guide. Furthermore, in another example, the reverse surface of the storage contains instructional materials for the use of infusion device including a quick reference guide or frequently asked questions.

In another example, the storage includes patient support resources, e.g., a place to write the date the spacer guide was opened, a tool to track how many times the guide has been used and/or how many uses the guide has left, and a tool to track injection site usage and site rotation across multiple infusions. The patient support resources on the storage can be tailored such that a patient receives different resources at different times. For example, it may be advantageous to provide one set of resources to new patients and a second set of resources to experienced patients.

Optionally, as shown in FIG. 25, an illustration 40 of the infusion site, e.g., abdomen, may be superimposed behind the needle guide storage location. Patients may be instructed to store their needle guide by placing it on the illustration 40 in the same orientation they just used. In this way, at the next dose patients will have a visual reminder of the orientation used in their prior dose.

Optionally, the storage may be designed for use as a work mat during infusion preparation. The storage material should then be easy to clean or sterilize so that the user may place sterile preparation components, e.g., vials, syringes, vial spikes, upon the storage. Optionally, the another surface of the storage may be designed for use as a work mat by including labelled areas for different preparation components.

Furthermore, the kit of the needle assembly may comprise groups of bandages as visual cue to prior needle placement. For example, the users are instructed to place the bandages on the injection sites after use such that the users can place the needle spacer arrangement away from the previous injection sites.

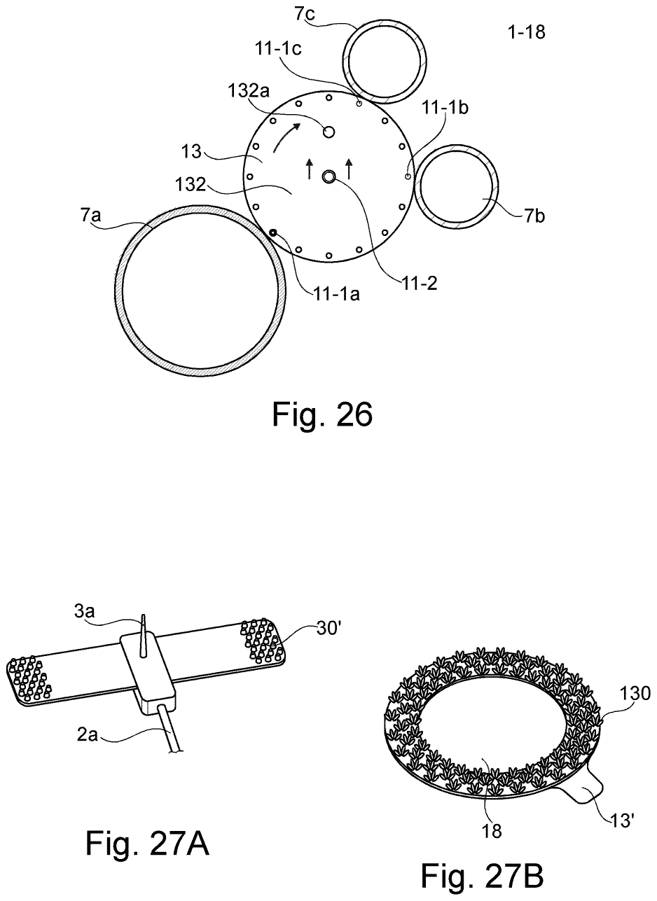

Optionally, the needle guide as disclosed in any example above is manufactured in a transparent semi-rigid material. Appropriate needle placement may be algorithmically generated to separate needles based on a medication regimen and patient parameters including but not limited to preferred injection site, e.g., abdomen or thigh, preferred side, e.g., left or right, anthropometric measurements, locations of anatomic references, e.g., the umbilicus, and information about improper injection locations, e.g., scar tissue. The outer profile of the needle guide and interfaces for needle assemblies can then be cut from stock material. Optionally, machine vision may be used to attach the needle into the needle guide. In one example, the needle guide comprises a movable cover configured to selectively cover the some of the openings/guide surfaces of the needle guide. For example, as shown in FIG. 26, the needle guide 13 comprises a rotational cover 132 selectively cover the some of the guide surfaces 11-1a, 11-ab, 11-1c of the needle guide 13. In one example, a guiding feature, e.g., colour codes or marks, can be used. In this example, the guide surfaces comprise the guiding feature. For example, the guide surfaces are coloured with different colours. In this example, the rotational cover 132 is configured to cover some guide surfaces and only allow the guide surfaces with the same colour exposed. In this example, the user is instructed to place the needle spacer elements to the matched colour.

In one example where the needle guide 13 is reusable and is configured to be attached to the skin in a position based on the reference point 11-2, e.g., the needle guide is configured to be centred on the umbilicus or knee, as mentioned above, the movable cover 132 is configured to guide the user to avoid inserting needles into sites that the tissue has not recovered yet. Thus, the advantage of injection sites rotation can be achieved. For example, the rotational cover as shown in FIG. 26 comprises an indication opening 132a configured to provide an indication to the user. In one example, the indication can be a series of letters ‘A, B, C . . . ’, the user is instructed to rotate the rotational cover 132 around the reference point 11-2 to the next letter after the injection or before carrying out a new injection. As a result, the guide surfaces 11-1a, 11-1b, 11-1c that received the needles and/or the needle spacer elements 7a, 7b, 7c for the previous injection will be covered by the rotational cover 132 such that the user cannot insert the needle into the sites of previous injection. In one example, the rotational cover can be an extra layer on top of the needle guide and being attached to the reference point.

The tubes from each needle assembly are routed into a bulk connector attached to the needle guide. Preferably, the tubes are routed between the skin and the needle guide to protect the lumens from snagging on the external environment. In some embodiments, the bulk connector can make to be able to collect more lumen connections than are required for a given patient's regimen, so that a single size of the bulk connector may be used for multiple medication regimens. Optionally, the bulk connector contains an internal mechanism that excess tube length may be wrapped around. This may be advantageous to reduce tangling of tube lumens while using a generic, mass-manufacturable needle assembly. Alternatively, the connector may be pre-assembled with tube lumens fully coiled and extended outwards during placement of the needle guide.

Optionally, the needle may be removed from the needle guide after administration so that the needles are discarded, and the guide is retained and reused.

In some embodiments, the needle guide is created as a service by a patient support program. This reusable needle guide may be decorated with branded drug information, logos, regimen indicia, or brand-specific colours. Alternatively, the visual design of the guide may be customized by the patient such as the inclusion of the patient's name, varying skin tones, or preferred colours.

In some embodiments, the needle guide is created for each patient in a clinical trial to ensure intra-personal consistency of needle placement across administrations. The reusable guide is optionally removed after needle insertion or worn throughout the infusion. This may be advantageous in decentralized trials to ensure the patient is placing needles correctly and consistently during self-administration at home. This may be especially advantageous for trials of combinations treatments where depot separation is critical.

The needle guide, and the needles situated therein, may take on numerous different outer shapes, depending on the needle design, the presence or absence of a needle inserter, the number of needles present, desired injection rotation schedule between injections, the volume of medicament to be infused at a given site through a given needle, nature of the medicines themselves, medication regimen, medication administration sequence(s), a desire to prevent mixing of medications at their respective injection sites, or based on results from pharmacokinetic/pharmacodynamic modelling or human clinical trial(s) of one or more medicaments. Further, each needle guide or needle spacer element need not be identical. Many permutations may be made available, or sequences of arrangements may be made available to a specific patient, as would be advantageous to support site rotation between injections.

In all examples in FIGS. 9-23, the needles may in some variations be provided with a respective needle spacer element. It should be noted that, the first needle inserter and the second needle inserter as mentioned below can be a part of the first needle spacer element and a part of the needle spacer element respectively as mentioned above.

The injection assemblies as being different combinations from above disclosed features/structures/elements provide solutions to ensure separations of multiple medicaments at their respective injection sites, or administration of a single medication across multiple injection sites, in a controlled, predictable manner. Additionally, different combinations can also provide different benefit to the user such as safety, e.g., make sure the needles will not hurt the tissue when the tube is accidentally pulled, comfortability, e.g., avoid repeating needle damage on the same skin tissue, and usability, e.g., variable way of user indications and wearable feature, that can make the injection assembly being easily used by the user. Some embodiments of the injection assemblies as being different combinations from above disclosed features/structures/elements are described below as examples.

In a first embodiment, the injection assembly provides a first needle and a second needle. In this embodiment, the first needle is an injection needle configured to penetrate through users' skin and is connected to a medicament delivery device via a first tube; the second needle is an injection needle configured to penetrate through users' skin is connected to a medicament delivery device via a second tube; the first needle is configured to deliver a first medicament from the medicament delivery device to a user; and the second needle is configured to deliver a second medicament from the medicament delivery device to the user. In one example, the first medicament and the second medicament are the same; alternatively, the first medicament and the second medicament are different from one another. The injection assembly further provides a needle spacing arrangement. The needle spacing arrangement comprises a first needle spacer element and a second needle spacer element. The first needle spacer element is configured to be attached to the first needle; and the second needle spacer element is configured to be attached to a second needle. The first needle spacer element has a first dimension, and the second needle spacer element has a second dimension; such that when the first needle spacer is placed adjacent to the second needle spacer, the first needle that is attached to the first needle spacer and the second needle that is attached to the second needle spacer are spaced apart from one another at a distance at which the medicament administered by the first needle and the second needle are prevented from mixing subcutaneously and/or is larger than a diameter of an expected first skin wheal formed subcutaneously as a result of the first predefined medicament administration and a diameter of an expected second skin wheal formed subcutaneously as a result of the second predefined medicament administration. The first needle spacer element and the second needle spacer element comprise magnetic material respectively. In one example, the first needle spacer element is configured to magnetically repel the second needle spacer element when the second needle spacer element is arranged within a predetermined distance from the first needle spacer element. In another example, the first needle spacer element comprises a skin marker and the second needle spacer element comprises a skin marker. As mentioned above, the skin marker is configured to mark the previous injection part on the skin of the user. The skin marker used in the first needle spacer element and/or the second needle spacer element can be adhesive or a stamp.

In a second embodiment, the injection assembly provides a first needle and a second needle. In the second embodiment, the first needle is a first soft cannula configured to be inserted into a user's body via an inserter; and the second needle is a second soft cannula configured to be inserted into the user's body via an inserter. Thus, in the second embodiment, the first needle is connected to a medicament delivery device directly or indirectly via a tube; the second needle is connected to a medicament delivery device directly, or indirectly via a tube; the first needle is configured to deliver a first medicament from the medicament delivery device to a user; and the second needle is configured to deliver a second medicament from the medicament delivery device to the user. In one example, the first medicament and the second medicament are the same; alternatively, the first medicament and the second medicament are different from one another. The injection assembly further provides a needle spacing arrangement. The needle spacing arrangement comprises a first needle spacer element and a second needle spacer element. The first needle spacer element is configured to be attached to the first needle; and the second needle spacer element is configured to be attached to a second needle. The first needle spacer element has a first dimension, and the second needle spacer element has a second dimension; such that when the first needle spacer is placed adjacent to the second needle spacer, the first needle that is attached to the first needle spacer and the second needle that is attached to the second needle spacer are spaced apart from one another at a distance at which the medicament administered by the first needle and the second needle are prevented from mixing subcutaneously and/or is larger than a diameter of an expected first skin wheal formed subcutaneously as a result of the first predefined medicament administration and a diameter of an expected second skin wheal formed subcutaneously as a result of the second predefined medicament administration. The first needle spacer element and the second needle spacer element comprise magnetic material respectively. In one example, the first needle spacer element is configured to magnetically repel the second needle spacer element when the second needle spacer element is arranged within a predetermined distance from the first needle spacer element. In another example, the first needle spacer comprises a skin marker and the second needle spacer element comprises a skin marker. As mentioned above, the skin marker is configured to mark the previous injection part on the skin of the user. The skin marker used in the first needle spacer element and/or the second needle spacer element can be adhesive or a stamp.

In a third embodiment, the injection assembly provides a first needle and a second needle. In the third embodiment, the first needle is a first soft cannula configured to be inserted into a user's body via an inserter; and the second needle is a second soft cannula configured to be inserted into the user's body via an inserter. In the third embodiment, the first needle is connected to a medicament delivery device directly or indirectly via a tube; the second needle is connected to a medicament delivery device directly, or indirectly via a tube; the first needle is configured to deliver a first medicament from the medicament delivery device to a user; and the second needle is configured to deliver a second medicament from the medicament delivery device to the user. In one example, the first medicament and the second medicament are the same; alternatively, the first medicament and the second medicament are different from one another. The injection assembly further provides a needle spacing arrangement. The needle spacing arrangement comprises a first needle spacer element and a second needle spacer element. The first needle spacer element is configured to be attached to the first needle; and the second needle spacer element is configured to be attached to a second needle. The first needle spacer element has a first dimension, and the second needle spacer element has a second dimension; such that when the first needle spacer is placed adjacent to the second needle spacer, the first needle that is attached to the first needle spacer and the second needle that is attached to the second needle spacer are spaced apart from one another at a distance at which the medicament administered by the first needle and the second needle are prevented from mixing subcutaneously and/or is larger than a diameter of an expected first skin wheal formed subcutaneously as a result of the first predefined medicament administration and a diameter of an expected second skin wheal formed subcutaneously as a result of the second predefined medicament administration. The first needle spacer element and the second needle spacer element comprise magnetic material respectively. In one example, the first needle spacer element is configured to magnetically repel the second needle spacer element when the second needle spacer element is arranged within a predetermined distance from the first needle spacer element. In this example, the injection assembly further provide a first cover and a second cover. The first cover is configured to cover the first needle during the medicament delivery operation, and the second cover is configured to cover the second needle during the medicament delivery operation. In one example, the first and the second covers are attached to the first and the second needle spacer elements respectively. In one example, the first and the second covers are attached to first needle and the second needle respectively such that the first needle and the second needle can be removed via removal of the first cover and the second cover.

In a fourth embodiment, the injection assembly provides a first needle and a second needle. In the fourth embodiment, the first needle is an injection needle configured to penetrate through users' skin and is connected to a medicament delivery device via a first tube; the second needle is an injection needle configured to penetrate through users' skin is connected to a medicament delivery device via a second tube; the first needle is configured to deliver a first medicament from the medicament delivery device to a user; and the second needle is configured to deliver a second medicament from the medicament delivery device to the user. In one example, the first medicament and the second medicament are the same; alternatively, the first medicament and the second medicament are different from one another. The injection assembly further provides a needle spacing arrangement. The needle spacing arrangement comprises a first needle spacer element and a second needle spacer element. The first needle spacer element is configured to be attached to the first needle; and the second needle spacer element is configured to be attached to a second needle. The first needle spacer element has a first dimension, and the second needle spacer element has a second dimension; such that when the first needle spacer is placed adjacent to the second needle spacer, the first needle that is attached to the first needle spacer and the second needle that is attached to the second needle spacer are spaced apart from one another at a distance at which the medicament administered by the first needle and the second needle are prevented from mixing subcutaneously and/or is larger than a diameter of an expected first skin wheal formed subcutaneously as a result of the first predefined medicament administration and a diameter of an expected second skin wheal formed subcutaneously as a result of the second predefined medicament administration. In the fourth embodiment, the first needle spacer element comprises a first tube retention fastener configured to fasten the first tube to the first needle spacer element; the second needle spacer comprises a second tube retention fastener configured to fasten the second tube to the second needle spacer element. As mentioned above, the retention fastener can be made of material has high Coefficient of friction, e.g., rubber, such that the tube is held by the tube retention fastener via friction. Alternatively, or additionally, the retention fastener is configured to engage with a counter tube retention fastener of the tube. For example, the retention fastener and the counter tube retention fastener can form a thread fastener or a bayonet fastener. In this example, the connection between the needle and the tube can be further secured.

In a fifth embodiment, the injection assembly provides a first needle and a second needle. In the fifth embodiment, the first needle is an injection needle configured to penetrate through users' skin and is connected to a medicament delivery device via a first tube; the second needle is an injection needle configured to penetrate through users' skin is connected to a medicament delivery device via a second tube; the first needle is configured to deliver a first medicament from the medicament delivery device to a user; and the second needle is configured to deliver a second medicament from the medicament delivery device to the user. In one example, the first medicament and the second medicament are the same; alternatively, the first medicament and the second medicament are different from one another. The injection assembly further provides a needle spacing arrangement. The needle spacing arrangement comprises a first needle spacer element, a second needle spacer element, and a needle guide. The first needle spacer element is configured to be attached to the first needle; and the second needle spacer element is configured to be attached to a second needle. The first needle spacer element is configured to be placed adjacent to a first guide surface of the needle guide; and the second needle spacer element is configured to be placed adjacent to a second guide surface of the needle guide. The first guide surface and the second guide surface is defined by different parts of the outer contour of the needle guide, such that the when the first needle spacer is placed adjacent to the first guide surface and the second needle spacer is placed adjacent to the second guide surface, the first needle that is attached to the first needle spacer and the second needle that is attached to the second needle spacer are spaced apart from one another at a distance at which the medicament administered by the first needle and the second needle are prevented from mixing subcutaneously and/or is larger than a diameter of an expected first skin wheal formed subcutaneously as a result of the first predefined medicament administration and a diameter of an expected second skin wheal formed subcutaneously as a result of the second predefined medicament administration. The first needle spacer element and the second needle spacer element comprise magnetic material respectively. In one example, the first needle spacer element is configured to magnetically repel the needle guide if the first needle spacer element is not placed adjacent to the first guide surface; and the second needle spacer element is configured to magnetically repel the needle guide if the second needle spacer element is not placed adjacent to the second guide surface.

In a sixth embodiment, the injection assembly provides a first needle and a second needle. In the sixth embodiment, the first needle comprises a first soft cannula configured to be inserted into a user's body via an inserter; and the second needle comprises a second soft cannula configured to be inserted into the user's body via an inserter. The first needle is configured to deliver a first medicament from the medicament delivery device to a user; and the second needle is configured to deliver a second medicament from the medicament delivery device to the user. In one example, the first medicament and the second medicament are the same; alternatively, the first medicament and the second medicament are different from one another. The injection assembly further provides a needle spacing arrangement comprising a needle guide. In this example, the first needle is configured to be inserted into the user's body via a first inserter; and the second needle is configured to be inserted into the user's body via a second inserter. In this embodiment, the first inserter is configured to be placed adjacent to a first guide surface of the needle guide to insert the first needle; and the second inserter is configured to be placed adjacent to a second guide surface of the needle guide to insert the second needle. The first guide surface and the second guide surface is defined by different parts of the outer contour of the needle guide, such that the when the first needle is inserted adjacent to the first guide surface and the second needle is inserted adjacent to the second guide surface, the first needle that is attached to the first needle spacer and the second needle that is attached to the second needle spacer are spaced apart from one another at a distance at which the medicament administered by the first needle and the second needle are prevented from mixing subcutaneously and/or is larger than a diameter of an expected first skin wheal formed subcutaneously as a result of the first predefined medicament administration and a diameter of an expected second skin wheal formed subcutaneously as a result of the second predefined medicament administration. The first inserter and the second inserter comprise magnetic material respectively. In one example, the first inserter is configured to magnetically repel the needle guide if the first inserter is not placed adjacent to the first guide surface; and the second inserter is configured to magnetically repel the needle guide if the second inserter is not placed adjacent to the second guide surface.

In a seventh embodiment, the injection assembly provides a first needle and a second needle. In the seventh embodiment, the first needle comprises a first soft cannula configured to be inserted into a user's body via an inserter; and the second needle comprises a second soft cannula configured to be inserted into the user's body via an inserter. The first needle is configured to deliver a first medicament from the medicament delivery device to a user; and the second needle is configured to deliver a second medicament from the medicament delivery device to the user. In one example, the first medicament and the second medicament are the same; alternatively, the first medicament and the second medicament are different from one another. The injection assembly further provides a needle spacing arrangement comprising a needle guide. In this example, the first needle is configured to be inserted into the user's body via a first inserter; and the second needle is configured to be inserted into the user's body via a second inserter. In this embodiment, the first inserter is configured to be placed adjacent to a first guide surface of the needle guide to insert the first needle; and the second inserter is configured to be placed adjacent to a second guide surface of the needle guide to insert the second needle. The needle guide comprises multiple opening. The first guide surface and the second guide surface is defined by different openings of the needle guide, such that the when the first needle is inserted adjacent to the first guide surface and the second needle is inserted adjacent to the second guide surface, the first needle that is attached to the first needle spacer and the second needle that is attached to the second needle spacer are spaced apart from one another at a distance at which the medicament administered by the first needle and the second needle are prevented from mixing subcutaneously and/or is larger than a diameter of an expected first skin wheal formed subcutaneously as a result of the first predefined medicament administration and a diameter of an expected second skin wheal formed subcutaneously as a result of the second predefined medicament administration. The first inserter and the second inserter comprise magnetic material respectively. In one example, the first inserter is configured to magnetically repel the needle guide if the first inserter is not placed adjacent to the first guide surface; and the second inserter is configured to magnetically repel the needle guide if the second inserter is not placed adjacent to the second guide surface.

In an eighth embodiment, the injection assembly provides a first needle and a second needle. In the eighth embodiment, the first needle is an injection needle configured to penetrate through users' skin and is connected to a medicament delivery device via a first tube; the second needle is an injection needle configured to penetrate through users' skin is connected to a medicament delivery device via a second tube; the first needle is configured to deliver a first medicament from the medicament delivery device to a user; and the second needle is configured to deliver a second medicament from the medicament delivery device to the user. In one example, the first medicament and the second medicament are the same; alternatively, the first medicament and the second medicament are different from one another. The injection assembly further provides a needle spacing arrangement. The needle spacing arrangement comprises a first needle spacer element, a second needle spacer element, and a needle guide. The first needle spacer element is configured to be attached to the first needle; and the second needle spacer element is configured to be attached to a second needle. The first needle spacer element is configured to be placed adjacent to a first guide surface of the needle guide; and the second needle spacer element is configured to be placed adjacent to a second guide surface of the needle guide. The first guide surface and the second guide surface is defined by different parts of the outer contour of the needle guide, such that the when the first needle spacer is placed adjacent to the first guide surface and the second needle spacer is placed adjacent to the second guide surface, the first needle that is attached to the first needle spacer and the second needle that is attached to the second needle spacer are spaced apart from one another at a distance at which the medicament administered by the first needle and the second needle are prevented from mixing subcutaneously and/or is larger than a diameter of an expected first skin wheal formed subcutaneously as a result of the first predefined medicament administration and a diameter of an expected second skin wheal formed subcutaneously as a result of the second predefined medicament administration. The first needle spacer element and the second needle spacer element are coloured with different colour code respectively. In one example, the colour code of the first needle spacer element is configured to match with a colour code of the first guide surface; and the colour code of the second needle spacer element is configured to match a colour code of the second guide surface. Alternatively, or additionally, the outer contour of the first needle spacer is shaped to be matched with the first guide surface; and the outer contour of the second needle spacer is shaped to be matched with the second guide surface.

In a ninth embodiment, the injection assembly provides a first needle and a second needle. In the ninth embodiment, the first needle is an injection needle configured to penetrate through users' skin and is connected to a medicament delivery device via a first tube; the second needle is an injection needle configured to penetrate through users' skin is connected to a medicament delivery device via a second tube; the first needle is configured to deliver a first medicament from the medicament delivery device to a user; and the second needle is configured to deliver a second medicament from the medicament delivery device to the user. In one example, the first medicament and the second medicament are the same; alternatively, the first medicament and the second medicament are different from one another. The injection assembly further provides a needle spacing arrangement. The needle spacing arrangement comprises a first needle spacer element, a second needle spacer element, and a needle guide. The first needle spacer element is configured to be attached to the first needle; and the second needle spacer element is configured to be attached to a second needle. The first needle spacer element is configured to be placed adjacent to a first guide surface of the needle guide; and the second needle spacer element is configured to be placed adjacent to a second guide surface of the needle guide. The needle guide as disclosed in the ninth embodiment comprises multiple openings. The first guide surface and the second guide surface is defined by different opening of the needle guide, such that the when the first needle spacer is placed adjacent to the first guide surface and the second needle spacer is placed adjacent to the second guide surface, the first needle that is attached to the first needle spacer and the second needle that is attached to the second needle spacer are spaced apart from one another at a distance at which the medicament administered by the first needle and the second needle are prevented from mixing subcutaneously and/or is larger than a diameter of an expected first skin wheal formed subcutaneously as a result of the first predefined medicament administration and a diameter of an expected second skin wheal formed subcutaneously as a result of the second predefined medicament administration. The first needle spacer element and the second needle spacer element are coloured with different colour code respectively. In one example, the colour code of the first needle spacer element is configured to match with a colour code of the first guide surface; and the colour code of the second needle spacer element is configured to match a colour code of the second guide surface. Alternatively, or additionally, the outer contour of the first needle spacer is shaped to be matched with the first guide surface; and the outer contour of the second needle spacer is shaped to be matched with the second guide surface.

In a tenth embodiment, the injection assembly provides a first needle and a second needle. In the tenth embodiment, the first needle comprises a first soft cannula configured to be inserted into a user's body via an inserter; and the second needle comprises a second soft cannula configured to be inserted into the user's body via an inserter. The first needle is configured to deliver a first medicament from the medicament delivery device to a user; and the second needle is configured to deliver a second medicament from the medicament delivery device to the user. In one example, the first medicament and the second medicament are the same; alternatively, the first medicament and the second medicament are different from one another. The injection assembly further provides a needle spacing arrangement comprising a needle guide. In this example, the first needle is configured to be inserted into the user's body via a first inserter; and the second needle is configured to be inserted into the user's body via a second inserter. In this embodiment, the first inserter is configured to be placed adjacent to a first guide surface of the needle guide to insert the first needle; and the second inserter is configured to be placed adjacent to a second guide surface of the needle guide to insert the second needle. The first guide surface and the second guide surface is defined by different parts of the outer contour of the needle guide, such that the when the first needle is inserted adjacent to the first guide surface and the second needle is inserted adjacent to the second guide surface, the first needle that is attached to the first needle spacer and the second needle that is attached to the second needle spacer are spaced apart from one another at a distance at which the medicament administered by the first needle and the second needle are prevented from mixing subcutaneously and/or is larger than a diameter of an expected first skin wheal formed subcutaneously as a result of the first predefined medicament administration and a diameter of an expected second skin wheal formed subcutaneously as a result of the second predefined medicament administration. The first inserter and the second inserter are colour with different colour code. In one example, the first inserter is configured to match with a colour code of the first guide surface; and the second inserter is configured to match with a colour code of the second guide surface. Alternatively, or additionally, the outer contour of the first inserter is shaped to be matched with the first guide surface; and the outer contour of the second inserter is shaped to be matched with the second guide surface.

In an eleventh embodiment, the injection assembly provides a first needle and a second needle. In the eleventh embodiment, the first needle comprises a first soft cannula configured to be inserted into a user's body via an inserter; and the second needle comprises a second soft cannula configured to be inserted into the user's body via an inserter. The first needle is configured to deliver a first medicament from the medicament delivery device to a user; and the second needle is configured to deliver a second medicament from the medicament delivery device to the user. In one example, the first medicament and the second medicament are the same; alternatively, the first medicament and the second medicament are different from one another. The injection assembly further provides a needle spacing arrangement comprising a needle guide. In this example, the first needle is configured to be inserted into the user's body via a first inserter; and the second needle is configured to be inserted into the user's body via a second inserter. In this embodiment, the first inserter is configured to be placed adjacent to a first guide surface of the needle guide to insert the first needle; and the second inserter is configured to be placed adjacent to a second guide surface of the needle guide to insert the second needle. The needle guide comprises multiple openings. The first guide surface and the second guide surface is defined by different openings of the needle guide, such that the when the first needle is inserted adjacent to the first guide surface and the second needle is inserted adjacent to the second guide surface, the first needle that is attached to the first needle spacer and the second needle that is attached to the second needle spacer are spaced apart from one another at a distance at which the medicament administered by the first needle and the second needle are prevented from mixing subcutaneously and/or is larger than a diameter of an expected first skin wheal formed subcutaneously as a result of the first predefined medicament administration and a diameter of an expected second skin wheal formed subcutaneously as a result of the second predefined medicament administration. The first inserter and the second inserter are colour with different colour code. In one example, the first inserter is configured to match with a colour code of the first guide surface; and the second inserter is configured to match with a colour code of the second guide surface.

In a twelfth embodiment, the injection assembly provides a first needle and a second needle. In the twelfth embodiment, the first needle is an injection needle configured to penetrate through users' skin and is connected to a medicament delivery device via a first tube; the second needle is an injection needle configured to penetrate through users' skin is connected to a medicament delivery device via a second tube; the first needle is configured to deliver a first medicament from the medicament delivery device to a user; and the second needle is configured to deliver a second medicament from the medicament delivery device to the user. In one example, the first medicament and the second medicament are the same; alternatively, the first medicament and the second medicament are different from one another. The injection assembly further provides a needle spacing arrangement. The needle spacing arrangement comprises a first needle spacer element, a second needle spacer element, and a needle guide. The first needle spacer element is configured to be attached to the first needle; and the second needle spacer element is configured to be attached to a second needle. The first needle spacer element is configured to be placed adjacent to a first guide surface of the needle guide; and the second needle spacer element is configured to be placed adjacent to a second guide surface of the needle guide. The first guide surface and the second guide surface is defined by different parts of the outer contour of the needle guide, such that the when the first needle spacer is placed adjacent to the first guide surface and the second needle spacer is placed adjacent to the second guide surface, the first needle that is attached to the first needle spacer and the second needle that is attached to the second needle spacer are spaced apart from one another at a distance at which the medicament administered by the first needle and the second needle are prevented from mixing subcutaneously and/or is larger than a diameter of an expected first skin wheal formed subcutaneously as a result of the first predefined medicament administration and a diameter of an expected second skin wheal formed subcutaneously as a result of the second predefined medicament administration. In the twelfth embodiment, the needle guide is configured to be reused by the user. In this embodiment, the needle guide comprises multiple sets of first guide surfaces and second guide surfaces. In this embodiment, the needle guide comprises a rotational cover 132 selectively cover the some of the guide surfaces. In this embodiment, the needle guide comprises a guiding feature, e.g., colour codes or marks, can be used. In this example, the guide surfaces comprise the guiding feature. For example, the guide surfaces are coloured with different colours. In this example, the rotational cover 132 is configured to cover some guide surfaces and only allow the guide surfaces with the same colour exposed. In this example, the user is instructed to place the needle spacer elements to the matched colour. As a result, the user not only uses the needle guide to avoid the potential skin wheat and/or subcutaneously medicament mixed, but also avoid injecting a skin tissue that is not yet recovering from the previous medicament delivery operation.

In a thirteenth embodiment, the injection assembly provides a first needle and a second needle. In the thirteenth embodiment, the first needle comprises a first soft cannula configured to be inserted into a user's body via an inserter; and the second needle comprises a second soft cannula configured to be inserted into the user's body via an inserter. The first needle is configured to deliver a first medicament from the medicament delivery device to a user; and the second needle is configured to deliver a second medicament from the medicament delivery device to the user. In one example, the first medicament and the second medicament are the same; alternatively, the first medicament and the second medicament are different from one another. The injection assembly further provides a needle spacing arrangement. The needle spacing arrangement comprises a first needle spacer element, a second needle spacer element, and a needle guide. In this example, the first needle is configured to be inserted into the user's body via a first inserter; and the second needle is configured to be inserted into the user's body via a second inserter. In this embodiment, the first inserter is configured to be placed adjacent to a first guide surface of the needle guide to insert the first needle; and the second inserter is configured to be placed adjacent to a second guide surface of the needle guide to insert the second needle. The needle guide comprises multiple openings. The first guide surface and the second guide surface is defined by different openings of the needle guide, such that the when the first needle is inserted adjacent to the first guide surface and the second needle is inserted adjacent to the second guide surface, the first needle that is attached to the first needle spacer and the second needle that is attached to the second needle spacer are spaced apart from one another at a distance at which the medicament administered by the first needle and the second needle are prevented from mixing subcutaneously and/or is larger than a diameter of an expected first skin wheal formed subcutaneously as a result of the first predefined medicament administration and a diameter of an expected second skin wheal formed subcutaneously as a result of the second predefined medicament administration. In the thirteenth embodiment, the needle guide is configured to be reused by the user. In this embodiment, the needle guide comprises multiple sets of first guide surfaces and second guide surfaces. In this embodiment, the needle guide comprises a rotational cover selectively cover the some of the guide surfaces. In this embodiment, the needle guide comprises a guiding feature, e.g., colour codes or marks, can be used. In this example, the guide surfaces comprise the guiding feature. For example, the guide surfaces are coloured with different colours. In this example, the rotational cover is configured to cover some guide surfaces and only allow the guide surfaces with the same colour exposed. In this example, the user is instructed to place the first inserter and the second inserter to the matched colour. As a result, the user not only uses the needle guide to avoid the potential skin wheat and/or subcutaneously medicament mixed, but also avoid injecting a skin tissue that is not yet recovering from the previous medicament delivery operation.

In a fourteenth embodiment, the injection assembly provides a first needle and a second needle. In the fourteenth embodiment, the first needle is an injection needle configured to penetrate through users' skin and is connected to a medicament delivery device via a first tube; the second needle is an injection needle configured to penetrate through users' skin is connected to a medicament delivery device via a second tube; the first needle is configured to deliver a first medicament from the medicament delivery device to a user; and the second needle is configured to deliver a second medicament from the medicament delivery device to the user. In one example, the first medicament and the second medicament are the same; alternatively, the first medicament and the second medicament are different from one another. The injection assembly further provides a needle spacing arrangement. The needle spacing arrangement comprises a first needle spacer element, a second needle spacer element, and a needle guide. The first needle spacer element is configured to be attached to the first needle; and the second needle spacer element is configured to be attached to a second needle. The first needle spacer element is configured to be placed adjacent to a first guide surface of the needle guide; and the second needle spacer element is configured to be placed adjacent to a second guide surface of the needle guide. The first guide surface and the second guide surface is defined by different parts of the outer contour of the needle guide; alternatively, multiple openings are arranged in the needle guide, and the first and the second guide surfaces are defined by the multiple openings, such that the when the first needle spacer is placed adjacent to the first guide surface and the second needle spacer is placed adjacent to the second guide surface, the first needle that is attached to the first needle spacer and the second needle that is attached to the second needle spacer are spaced apart from one another at a distance at which the medicament administered by the first needle and the second needle are prevented from mixing subcutaneously and/or is larger than a diameter of an expected first skin wheal formed subcutaneously as a result of the first predefined medicament administration and a diameter of an expected second skin wheal formed subcutaneously as a result of the second predefined medicament administration. In embodiment, the needle guide is configured to be worn by the user rather than placing onto user's skin via adhesive such that the user experience can be increased. In this embodiment, the needle guide comprises a wearable feature, e.g., a belt, a should stripe, a neck stripe, a belt clip, a leg sleeve, or a body sleeve. The needle guide may comprise an anatomical reference point, e.g., an opening that is configured to be lined up with the knee or the umbilicus. Alternatively, the needle guide can be design as a vest, in this example, the anatomical reference point is not necessary as the guide surfaces are located properly once the user wears the needle guide. In this embodiment, the needle guide can be worn by the user during the medicament delivery operation or can be removed from the user's body after the first and the second needles are placed on the skin.

In a fifteenth embodiment, the injection assembly provides a first needle and a second needle. In the fifteenth embodiment, the first needle comprises a first soft cannula configured to be inserted into a user's body via an inserter; and the second needle comprises a second soft cannula configured to be inserted into the user's body via an inserter. The first needle is configured to deliver a first medicament from the medicament delivery device to a user; and the second needle is configured to deliver a second medicament from the medicament delivery device to the user.

In one example, the first medicament and the second medicament are the same; alternatively, the first medicament and the second medicament are different from one another. The injection assembly further provides a needle spacing arrangement comprising a needle guide. In this example, the first needle is configured to be inserted into the user's body via a first inserter; and the second needle is configured to be inserted into the user's body via a second inserter. In this embodiment, the first inserter is configured to be placed adjacent to a first guide surface of the needle guide to insert the first needle; and the second inserter is configured to be placed adjacent to a second guide surface of the needle guide to insert the second needle. The needle guide comprises multiple openings. The first guide surface and the second guide surface is defined by different openings of the needle guide, alternatively, the first and the second guide surfaces can be defined by different parts of the outer contour of the needle guide, such that the when the first needle is inserted adjacent to the first guide surface and the second needle is inserted adjacent to the second guide surface, the first needle that is attached to the first needle spacer and the second needle that is attached to the second needle spacer are spaced apart from one another at a distance at which the medicament administered by the first needle and the second needle are prevented from mixing subcutaneously and/or is larger than a diameter of an expected first skin wheal formed subcutaneously as a result of the first predefined medicament administration and a diameter of an expected second skin wheal formed subcutaneously as a result of the second predefined medicament administration. In embodiment, the needle guide is configured to be worn by the user rather than placing onto user's skin via adhesive such that the user experience can be increased. In this embodiment, the needle guide comprises a wearable feature, e.g., a belt, a should stripe, a neck stripe, a belt clip, a leg sleeve, or a body sleeve. The needle guide may comprise an anatomical reference point, e.g., an opening that is configured to be lined up with the knee or the umbilicus. Alternatively, the needle guide can be design as a vest, in this example, the anatomical reference point is not necessary as the guide surfaces are located properly once the user wears the needle guide. In this embodiment, the needle guide can be worn by the user during the medicament delivery operation or can be removed from the user's body after the first and the second needles are placed on the skin.

In a sixteenth embodiment, the injection assembly provides a first needle and a second needle. In the sixteenth embodiment, the first needle is an injection needle configured to penetrate through users' skin and is connected to a medicament delivery device via a first tube; the second needle is an injection needle configured to penetrate through users' skin is connected to a medicament delivery device via a second tube; the first needle is configured to deliver a first medicament from the medicament delivery device to a user; and the second needle is configured to deliver a second medicament from the medicament delivery device to the user. In one example, the first medicament and the second medicament are the same; alternatively, the first medicament and the second medicament are different from one another. The injection assembly further provides a needle spacing arrangement. The needle spacing arrangement comprises a first needle spacer element, a second needle spacer element, and a needle guide. The first needle spacer element is configured to be attached to the first needle; and the second needle spacer element is configured to be attached to a second needle. The first needle spacer element is configured to be placed adjacent to a first guide surface of the needle guide; and the second needle spacer element is configured to be placed adjacent to a second guide surface of the needle guide. The first guide surface and the second guide surface is defined by different parts of the outer contour of the needle guide; alternatively, multiple openings are arranged in the needle guide, and the first and the second guide surfaces are defined by the multiple openings, such that the when the first needle spacer is placed adjacent to the first guide surface and the second needle spacer is placed adjacent to the second guide surface, the first needle that is attached to the first needle spacer and the second needle that is attached to the second needle spacer are spaced apart from one another at a distance at which the medicament administered by the first needle and the second needle are prevented from mixing subcutaneously and/or is larger than a diameter of an expected first skin wheal formed subcutaneously as a result of the first predefined medicament administration and a diameter of an expected second skin wheal formed subcutaneously as a result of the second predefined medicament administration. In this embodiment, the needle guide is configured to be worn by the user rather than placing onto user's skin via adhesive such that the user experience can be increased. In this embodiment, the needle guide comprises a wearable feature, e.g., a belt, a should stripe, a neck stripe, a belt clip, a leg sleeve, or a body sleeve. The needle guide may comprise an anatomical reference point, e.g., an opening that is configured to be lined up with the knee or the umbilicus. Alternatively, the needle guide can be design as a vest, in this example, the anatomical reference point is not necessary as the guide surfaces are located properly once the user wears the needle guide. Furthermore, in the sixteenth embodiment, the needle guide is configured to be worn by the user during medicament delivery operation. In this embodiment, the first and the second needle spacer elements comprise counter needle mounts respectively. In this embodiment, the needle guide comprises needle mounts, adjacent to the first and the second guide surfaces, such that the first needle is configured to be attached to the user's body via the needle mount being attached to the counter needle mount that is adjacent to the first guide surface; and the second needle is configured to be attached to the user's body via the needle mount being attached to the counter needle mount that is adjacent to the second guide surface. In this embodiment, the counter needle mount of the first needle spacer element and the needle mount around the first guide surface formed a hook-and-loop fastener; and the second needle mount of the second needle spacer element and the needle mount around the second guide surface formed a hook-and-loop fastener. Alternatively, or additionally, the injection assembly further comprises a cover configured to cover the guide surfaces of the needle guide during medicament delivery operation, such that the first and the second needles can also be covered.

In a seventeenth embodiment, the injection assembly provides a first needle and a second needle. In the fifteenth embodiment, the first needle comprises a first soft cannula configured to be inserted into a user's body via an inserter; and the second needle comprises a second soft cannula configured to be inserted into the user's body via an inserter. The first needle is configured to deliver a first medicament from the medicament delivery device to a user; and the second needle is configured to deliver a second medicament from the medicament delivery device to the user. In one example, the first medicament and the second medicament are the same; alternatively, the first medicament and the second medicament are different from one another. The injection assembly further provides a needle spacing arrangement comprising a needle guide. In this example, the first needle is configured to be inserted into the user's body via a first inserter; and the second needle is configured to be inserted into the user's body via a second inserter. In this embodiment, the first inserter is configured to be placed adjacent to a first guide surface of the needle guide to insert the first needle; and the second inserter is configured to be placed adjacent to a second guide surface of the needle guide to insert the second needle. The needle guide comprises multiple openings. The first guide surface and the second guide surface is defined by different openings of the needle guide, alternatively, the first and the second guide surfaces can be defined by different parts of the outer contour of the needle guide, such that the when the first needle is inserted adjacent to the first guide surface and the second needle is inserted adjacent to the second guide surface, the first needle that is attached to the first needle spacer and the second needle that is attached to the second needle spacer are spaced apart from one another at a distance at which the medicament administered by the first needle and the second needle are prevented from mixing subcutaneously and/or is larger than a diameter of an expected first skin wheal formed subcutaneously as a result of the first predefined medicament administration and a diameter of an expected second skin wheal formed subcutaneously as a result of the second predefined medicament administration. In embodiment, the needle guide is configured to be worn by the user rather than placing onto user's skin via adhesive such that the user experience can be increased. In this embodiment, the needle guide comprises a wearable feature, e.g., a belt, a should stripe, a neck stripe, a belt clip, a leg sleeve, or a body sleeve. The needle guide may comprise an anatomical reference point, e.g., an opening that is configured to be lined up with the knee or the umbilicus. Alternatively, the needle guide can be design as a vest, in this example, the anatomical reference point is not necessary as the guide surfaces are located properly once the user wears the needle guide. Furthermore, in this embodiment, the needle guide is configured to be worn by the user during medicament delivery operation, the first and the second needle inserters comprise counter needle mounts respectively. In this embodiment, the needle guide comprises needle mounts, adjacent to the first and the second guide surfaces, such that the first and the second inserters are secured to the needle guide during needle insertion and/or during medicament delivery operation. In this embodiment, the needle mounts and the counter needle mounts formed hook-and-loop fasteners. Alternatively, or additionally, the injection assembly further comprises a cover configured to cover the guide surfaces of the needle guide during medicament delivery operation, such that the first and the second needles can also be covered.

In an eighteenth embodiment, the injection assembly provides a first needle and a second needle. In the eighteenth embodiment, the first needle is an injection needle configured to penetrate through users' skin and is connected to a medicament delivery device via a first tube; the second needle is an injection needle configured to penetrate through users' skin is connected to a medicament delivery device via a second tube; the first needle is configured to deliver a first medicament from the medicament delivery device to a user; and the second needle is configured to deliver a second medicament from the medicament delivery device to the user. In one example, the first medicament and the second medicament are the same; alternatively, the first medicament and the second medicament are different from one another. The injection assembly further provides a needle spacing arrangement. The needle spacing arrangement comprises a first needle spacer element, a second needle spacer element, and a needle guide. The first needle spacer element is configured to be attached to the first needle; and the second needle spacer element is configured to be attached to a second needle. The first needle spacer element is configured to be placed adjacent to a first guide surface of the needle guide; and the second needle spacer element is configured to be placed adjacent to a second guide surface of the needle guide. The first guide surface and the second guide surface is defined by different parts of the outer contour of the needle guide; alternatively, multiple openings are arranged in the needle guide, and the first and the second guide surfaces are defined by the multiple openings, such that the when the first needle spacer is placed adjacent to the first guide surface and the second needle spacer is placed adjacent to the second guide surface, the first needle that is attached to the first needle spacer and the second needle that is attached to the second needle spacer are spaced apart from one another at a distance at which the medicament administered by the first needle and the second needle are prevented from mixing subcutaneously and/or is larger than a diameter of an expected first skin wheal formed subcutaneously as a result of the first predefined medicament administration and a diameter of an expected second skin wheal formed subcutaneously as a result of the second predefined medicament administration. In embodiment, the needle guide is configured to be worn by the user rather than placing onto user's skin via adhesive such that the user experience can be increased. In this embodiment, the needle guide comprises a wearable feature, e.g., a belt, a should stripe, a neck stripe, a belt clip, a leg sleeve, or a body sleeve. The needle guide may comprise an anatomical reference point, e.g., an opening that is configured to be lined up with the knee or the umbilicus. Alternatively, the needle guide can be design as a vest, in this example, the anatomical reference point is not necessary as the guide surfaces are located properly once the user wears the needle guide. Furthermore, in the eighteenth embodiment, the needle guide is configured to be worn by the user during medicament delivery operation. In this embodiment, the first and the second needle spacer elements comprise counter needle mounts respectively. In this embodiment, the needle guide comprises needle mounts, adjacent to the first and the second guide surfaces, such that the first needle is configured to be attached to the user's body via the needle mount o being attached to the counter needle mount that is adjacent to the first guide surface; and the second needle is configured to be attached to the user's body via the needle mount being attached to the counter needle mount that is adjacent to the second guide surface. The needle mounts and the counter needle mounts can be adhesive or can formed a hook-and-loop fastener. Additionally, the first needle spacer element comprises a first tube retention fastener configured to fasten the first tube to the first needle spacer element; the second needle spacer comprises a second tube retention fastener configured to fasten the second tube to the second needle spacer element. As mentioned above, the retention fastener can be made of material has high Coefficient of friction, e.g., rubber, such that the tube is held by the tube retention fastener via friction. Alternatively, or additionally, the retention fastener is configured to engage with a counter tube retention fastener of the tube. For example, the retention fastener and the counter tube retention fastener can form a thread fastener or a bayonet fastener. In this example, the connection between the needle and the tube can be further secured.

In a nineteenth embodiment, the injection assembly provides a first needle and a second needle. In the nineteenth embodiment, the first needle is an injection needle configured to penetrate through users' skin and is connected to a medicament delivery device via a first tube; the second needle is an injection needle configured to penetrate through users' skin is connected to a medicament delivery device via a second tube; the first needle is configured to deliver a first medicament from the medicament delivery device to a user; and the second needle is configured to deliver a second medicament from the medicament delivery device to the user. In one example, the first medicament and the second medicament are the same; alternatively, the first medicament and the second medicament are different from one another. The injection assembly further provides a needle spacing arrangement. The needle spacing arrangement comprises a first needle spacer element, a second needle spacer element, and a needle guide. The first needle spacer element is configured to be attached to the first needle; and the second needle spacer element is configured to be attached to a second needle. The first needle spacer element is configured to be placed adjacent to a first guide surface of the needle guide; and the second needle spacer element is configured to be placed adjacent to a second guide surface of the needle guide. The first guide surface and the second guide surface is defined by different parts of the outer contour of the needle guide; alternatively, multiple openings are arranged in the needle guide, and the first and the second guide surfaces are defined by the multiple openings, such that the when the first needle spacer is placed adjacent to the first guide surface and the second needle spacer is placed adjacent to the second guide surface, the first needle that is attached to the first needle spacer and the second needle that is attached to the second needle spacer are spaced apart from one another at a distance at which the medicament administered by the first needle and the second needle are prevented from mixing subcutaneously and/or is larger than a diameter of an expected first skin wheal formed subcutaneously as a result of the first predefined medicament administration and a diameter of an expected second skin wheal formed subcutaneously as a result of the second predefined medicament administration. In this embodiment, the first needle spacer comprises a skin marker and the second needle spacer element comprises a skin marker. As mentioned above, the skin marker is configured to mark the previous injection part on the skin of the user. The skin marker used in the first needle spacer element and/or the second needle spacer element can be adhesive or a stamp.

In a twentieth embodiment, the injection assembly provides a first needle and a second needle. In the twentieth embodiment, the first needle comprises a first soft cannula configured to be inserted into a user's body via an inserter; and the second needle comprises a second soft cannula configured to be inserted into the user's body via an inserter. The first needle is configured to deliver a first medicament from the medicament delivery device to a user; and the second needle is configured to deliver a second medicament from the medicament delivery device to the user. In one example, the first medicament and the second medicament are the same; alternatively, the first medicament and the second medicament are different from one another. The injection assembly further provides a needle spacing arrangement comprising a needle guide. In this example, the first needle is configured to be inserted into the user's body via a first inserter; and the second needle is configured to be inserted into the user's body via a second inserter. In this embodiment, the first inserter is configured to be placed adjacent to a first guide surface of the needle guide to insert the first needle; and the second inserter is configured to be placed adjacent to a second guide surface of the needle guide to insert the second needle. The needle guide comprises multiple openings. The first guide surface and the second guide surface is defined by different openings of the needle guide, alternatively, the first and the second guide surfaces can be defined by different parts of the outer contour of the needle guide, such that the when the first needle is inserted adjacent to the first guide surface and the second needle is inserted adjacent to the second guide surface, the first needle that is attached to the first needle spacer and the second needle that is attached to the second needle spacer are spaced apart from one another at a distance at which the medicament administered by the first needle and the second needle are prevented from mixing subcutaneously and/or is larger than a diameter of an expected first skin wheal formed subcutaneously as a result of the first predefined medicament administration and a diameter of an expected second skin wheal formed subcutaneously as a result of the second predefined medicament administration. In this embodiment, the first inserter comprises a skin marker and the second inserter comprises a skin marker. As mentioned above, the skin marker is configured to mark the previous injection part on the skin of the user. The skin marker used in the first inserter and/or the second inserter can be adhesive or a stamp.

The injection assemblies described herein can be used for the treatment and/or prophylaxis of one or more of many different types of disorders. Exemplary disorders include, but are not limited to: rheumatoid arthritis, inflammatory bowel diseases (e.g. Crohn's disease and ulcerative colitis), hypercholesterolaemia, diabetes (e.g. type 2 diabetes), psoriasis, migraines, multiple sclerosis, anaemia, lupus, atopic dermatitis, asthma, nasal polyps, acute hypoglycaemia, obesity, anaphylaxis and allergies. Exemplary types of drugs that could be included in the medicament delivery devices described herein include, but are not limited to, small molecules, hormones, cytokines, blood products, antibodies, antibody-drug conjugates, bispecific antibodies, proteins, fusion proteins, peptibodies, polypeptides, pegylated proteins, protein fragments, protein analogues, protein variants, protein precursors, chimeric antigen receptor T cell therapies, cell or gene therapies, oncolytic viruses, or immunotherapies. exemplary drugs that could be included in the injection assemblies described herein include, but are not limited to (with non-limiting examples of relevant disorders in brackets): etanercept (rheumatoid arthritis, inflammatory bowel diseases (e.g. Crohn's disease and ulcerative colitis)), evolocumab (hypercholesterolaemia), exenatide (type 2 diabetes), secukinumab (psoriasis), erenumab (migraines), alirocumab (rheumatoid arthritis), methotrexate (amethopterin) (rheumatoid arthritis), tocilizumab (rheumatoid arthritis), interferon beta-1a (multiple sclerosis), sumatriptan (migraines), adalimumab (rheumatoid arthritis), darbepoetin alfa (anaemia), belimumab (lupus), peginterferon beta-1a′ (multiple sclerosis), sarilumab (rheumatoid arthritis), semaglutide (type 2 diabetes, obesity), dupilumab (atopic dermatitis, asthma, nasal polyps, allergies), glucagon (acute hypoglycaemia), epinephrine (anaphylaxis), insulin (diabetes), atropine and vedolizumab (inflammatory bowel diseases (e.g. Crohn's disease and ulcerative colitis)), immunoglobulins (primary immune deficiencies), ipilimumab, nivolumab, pembrolizumab, atezolizumab, durvalumab, avelumab, cemiplimab, rituximab, trastuzumab, ado-trastuzumab emtansine, fam-trastuzumab deruxtecan-nxki, pertuzumab, transtuzumab-pertuzumab, alemtuzumab, belantamab mafodotin-blmf, bevacizumab, blinatumomab, brentuximab vedotin, cetuximab, daratumumab, elotuzumab, gemtuzumab ozogamicin, 90-Yttrium-ibritumomab tiuxetan, isatuximab, mogamulizumab, moxetumomab pasudotox, obinutuzumab, ofatumumab, olaratumab, panitumumab, polatuzumab vedotin, ramucirumab, sacituzumab govitecan, tafasitamab, or margetuximab. Pharmaceutical formulations including, but not limited to, any drug described herein are also contemplated for use in the medicament delivery devices described herein, for example pharmaceutical formulations comprising a drug as listed herein (or a pharmaceutically acceptable salt of the drug) and a pharmaceutically acceptable carrier. Pharmaceutical formulations comprising a drug as listed herein (or a pharmaceutically acceptable salt of the drug) may include one or more other active ingredients or may be the only active ingredient present. Pharmaceutical formulations may also include separately administered or co-formulated dispersion enhancers, such as hyaluronidase.

Exemplary drugs that could be included in the medicament delivery devices described herein include, but are not limited to, an immuno-oncology or bio-oncology medications such as immune checkpoints, cytokines, chemokines, clusters of differentiation, interleukins, integrins, growth factors, enzymes, signaling proteins, pro-apoptotic proteins, anti-apoptotic proteins, T-cell receptors, B-cell receptors, or costimulatory proteins.

Exemplary drugs that could be included in the medicament delivery devices described herein include, but are not limited to, those exhibiting a proposed mechanism of action, such as HER-2 receptor modulators, interleukin modulators, interferon modulators, CD38 modulators, CD22 modulators, CCR4 modulators, VEGF modulators, EGFR modulators, CD79b modulators, Trop-2 modulators, CD52 modulators, BCMA modulators, PDGFRA modulators, SLAMF7 modulators, PD-1/PD-L1 inhibitors/modulators, B-lymphocyte antigen CD19 inhibitors, B-lymphocyte antigen CD20 modulators, CD3 modulators, CTLA-4 inhibitors, TIM-3 modulators, VISTA modulators, INDO inhibitors, LAG3 (CD223) antagonists, CD276 antigen modulators, CD47 antagonists, CD30 modulators, CD73 modulators, CD66 modulators, CDw137 agonists, CD158 modulators, CD27 modulators, CD58 modulators, CD80 modulators, CD33 modulators, APRIL receptor modulators, HLA antigen modulators, EGFR modulators, B-lymphocyte cell adhesion molecule modulators, CDw123 modulators, Erbb2 tyrosine kinase receptor modulators, mesothelin modulators, HAVCR2 antagonists, NY-ESO-1 OX40 receptor agonist modulators, adenosine A2 receptors, ICOS modulators, CD40 modulators, TIL therapies, or TCR therapies. Exemplary drugs that could be included in the medicament delivery devices described herein include, but are not limited to, a multi-medication treatment regimen such as AC, Dose-Dense AC, TCH, GT, EC, TAC, TC, TCHP, CMF, FOLFOX, mFOLFOX6, mFOLFOX7, FOLFCIS, CapeOx, FLOT, DCF, FOLFIRI, FOLFIRINOX, FOLFOXIRI, IROX, CHOP, R-CHOP, RCHOP-21, Mini-CHOP, Maxi-CHOP, VR-CAP, Dose-Dense CHOP, EPOCH, Dose-Adjusted EPOCH, R-EPOCH, CODOX-M, IVAC, HyperCVAD, R-HyperCVAD, SC-EPOCH-RR, DHAP, ESHAP, GDP, ICE, MINE, CEPP, CDOP, GemOx, CEOP, CEPP, CHOEP, CHP, GCVP, DHAX, CALGB 8811, HIDAC, MOPAD, 7+3, 5+2, 7+4, MEC, CVP, RBAC500, DHA-Cis, DHA-Ca, DHA-Ox, RCVP, RCEPP, RCEOP, CMV, DDMVAC, GemFLP, ITP, VIDE, VDC, VAI, VDC-IE, MAP, PCV, FCR, FR, PCR, HDMP, OFAR, EMA/CO, EMA/EP, EP/EMA, TP/TE, BEP, TIP, VIP, TPEx, ABVD, BEACOPP, AVD, Mini-BEAM, IGEV, C-MOPP, GCD, GEMOX, CAV, DT-PACE, VTD-PACE, DCEP, ATG, VAC, VeIP, OFF, GTX, CAV, AD, MAID, AIM, VAC-IE, ADOC, or PE.

Exemplary drugs that could be included in the medicament delivery devices described herein include, but are not limited to, those used for chemotherapy, such as an alkylating agent, plant alkaloid, antitumor antibiotic, antimetabolite, or topoisomerase inhibitor, enzyme, retinoid, or corticosteroid. Exemplary chemotherapy drugs include, by way of example but not limitation, 5-fluorouracil, cisplatin, carboplatin, oxaliplatin, doxorubicin, daunorubicin, idarubicin, epirubicin, paclitaxel, docetaxel, cyclophosphamide, ifosfamide, azacitidine, decitabine, bendamustine, bleomycin, bortezomib, busulfan, cabazitaxel, carmustine, cladribine, cytarabine, dacarbazine, etoposide, fludarabine, gemcitabine, irinotecan, leucovorin, melphalan, methotrexate, pemetrexed, mitomycin, mitoxantrone, temsirolimus, topotecan, valrubicin, vincristine, vinblastine, or vinorelbine.

The inventive concept has mainly been described above with reference to a few examples. However, as is readily appreciated by a person skilled in the art, other embodiments than the ones disclosed above are equally possible within the scope of the inventive concept, as defined by the appended claims.

Some other aspects of the invention are disclosed in the following clauses.

1. An injection assembly (1-1; 1-2; 1-3; 1-4; 1-5; 1-6; 1-7; 1-8; 1-9; 1-10; 1-11; 1-12; 1-13; 1-14; 1-15; 1-16; 1-17; 1-18; 1-19) for medicament administration with a plurality of needles (3a, 3b, 3c, 3d), the injection assembly (1-1; 1-2; 1-3; 1-4; 1-5; 1-6; 1-7; 1-8; 1-9; 1-10; 1-11; 1-12; 1-13; 1-14; 1-15; 1-16; 1-17; 1-18; 1-19) comprising:
a first needle (3a) for a first predefined medicament administration,
   a second needle (3b) for a second predefined medicament administration, and
a needle spacer arrangement (5-1; 5-2; 5-3) configured to be removably attached to the skin of a user,
   the needle spacer arrangement (5-1; 5-2; 5-3) being configured to provide a spacing between the first needle (3a) and the second needle (3b),
   wherein the spacing is predetermined based on at least one of characteristics of the medicament, characteristics of infusion parameters for each medicament, physiologic parameters, or combinations thereof.

2. The injection assembly (1-1; 1-2; 1-3; 1-4; 1-5; 1-6; 1-7; 1-18) as described in clause 1, wherein the spacing is sized such that medicament administered by the first needle (3a) and the second needle (3b) are prevented from mixing subcutaneously and/or is larger than a diameter of an expected first skin wheal (9a) formed subcutaneously as a result of the first predefined medicament administration and a diameter of an expected second skin wheal (9b) formed subcutaneously as a result of the second predefined medicament administration.

3. The injection assembly (1-1; 1-2; 1-3; 1-4; 1-5; 1-6; 1-7; 1-18) as described in clause 2, wherein the spacing is larger than a diameter of an expected first skin wheal (9a) formed subcutaneously as a result of the first predefined medicament administration and a diameter of an expected second skin wheal (9b) formed subcutaneously as a result of the second predefined medicament administration.

4. The injection assembly (1-1; 1-2; 1-3; 1-4; 1-5; 1-6; 1-7; 1-18) as described in any one of the preceding clauses, wherein the amount of the first predefined medicament administration is above 1 ml.

5. The injection assembly (1-1; 1-2; 1-3; 1-4; 1-5; 1-6; 1-7; 1-18) as described in clause 4, wherein the amount of the first predefined medicament administration is above 3 ml.

6. The injection assembly (1-1; 1-2; 1-3; 1-4; 1-5; 1-6; 1-7; 1-18) as described in any one of the preceding clauses, wherein the amount of the second predefined medicament administration is above 1 ml.

7. The injection assembly (1-1; 1-2; 1-3; 1-4; 1-5; 1-6; 1-7; 1-18) as described in clause 6, wherein the amount of the second predefined medicament administration is above 3 ml.

8 The injection assembly (1-1; 1-2; 1-3; 1-4; 1-5; 1-6; 1-7; 1-18) as described in any one of clauses 3-5, wherein the sum of the amount of the first predefined medicament administration and the amount of the second predefined medicament administration is above 10 ml.

9. The injection assembly (1-1; 1-2; 1-3; 1-4; 1-5; 1-6; 1-7; 1-18) as described in any one of preceding clauses, wherein the molecular weight of the first predefined medicament is above 10 kilodaltons (kDa).

10. The injection assembly (1-1; 1-2; 1-3; 1-4; 1-5; 1-6; 1-7; 1-18) as described in any one of preceding clauses, wherein the molecular weight of the second predefined medicament is above 10 kilodaltons (kDa).

11. The injection assembly (1-1; 1-2; 1-3; 1-4; 1-5; 1-6; 1-7; 1-18) as described in any one of preceding clauses, wherein the needle spacer arrangement (5-1; 5-2; 5-3) comprises a first needle spacer element (7a) and a second needle spacer element (7b) physically separate from the first needle spacer element (7a).

12. The injection assembly as described in any one of preceding clauses, wherein the first needle spacer element comprises a first needle inserter configured to insert the first needle (3a) into the skin of the user; and the second needle spacer element comprises a second needle inserter configured to insert the second needle into the skin of the user.

13. The injection assembly (1-1; 1-2; 1-3; 1-4; 1-5; 1-6; 1-7; 1-18) as described in clause 1, wherein the first needle (3a) is mounted to the first needle spacer element (7a; 7a′; 7a″; 7a′″; 7a′″) and the second needle (3b) is mounted to the second needle spacer element (7b; 7b′″).

14. The injection assembly (1-2; 1-3; 1-4; 1-6; 1-7; 1-8; 1-9; 1-10; 1-11; 1-12; 1-13; 1-14; 1-15; 1-16; 1-17; 1-18; 1-19) as described in any of the preceding clauses, wherein the needle spacer arrangement (5-2; 5-3) comprises a needle guide (13; 13′; 13″) configured to be removably attached to the skin of the user.

15. The injection assembly (1-2; 1-3; 1-4; 1-18) as described in clause 12 when dependent on any one of clauses 2-5, wherein the needle guide (13) has an outer contour (11-1) which forms a first guide surface (11-1a) and a second guide surface (11-1b), the first guide surface (11-1a) being adapted to an outer contour of the first needle spacer element (7a; 7a′; 7a″; 7a′″; 7a′″) for positioning the first needle spacer element (7a; 7a′; 7a″; 7a′″; 7a′″) relative to the needle guide (13), and the second guide surface (11-1b) being adapted to an outer contour of the second needle spacer element (7b; 7b′″) for positioning the second needle spacer element (7b; 7b′″) relative to the needle guide (13).

16. The injection assembly (1-2) as described in clause 15, wherein each of the first guide surface (11-1a) and the second guide surface (11-1b) is configured to mate with a respective one of the first needle spacer element (7a; 7a′; 7a″; 7a′″; 7a′″) and the second needle spacer element (7b; 7b′″).

17. The injection assembly (1-3) as described in clause 15, wherein each of the first guide surface (11-1a) and the second guide surface is noncongruent with a respective one of the first needle spacer element (7a; 7a′; 7a″; 7a′″; 7a′″) and the second needle spacer element (7b; 7b′″).

18. The injection assembly (1-3) as described in clause 17, wherein the first guide surface (11-1a) is colour matched with the first needle spacer element (7a; 7a′; 7a″; 7a′″; 7a′″), and the second guide surface (11-1b) is colour matched with the second needle spacer element (7b; 7b′″).

19. The injection assembly as described in clause 14, wherein the needle guide (13′) has a first opening configured to receive the first needle (3a), and a second opening configured to receive the second needle (3b).

20. The injection assembly as described in clause 19, wherein the needle guide (13′) comprises a first needle mount adjacent to the first opening, and a second needle mount adjacent to the second opening; and wherein the first needle (3a) and the second needle (3b) are configured to be releasably mounted to the first and the second needle mounts of the needle guide (13′).

21. The injection assembly as described in clause 20, wherein the needle guide (13) comprises multiple openings for receiving multiple needles respectively; wherein the needle guide (13) comprises multiple needle mounts adjacent to the multiple openings respectively; and wherein at least one needle mount of the multiple needle mounts comprises an identifying feature to differentiate the at least one needle mount from the rest of the multiple needle mounts.

22. The injection assembly as described in clause 12 when dependent on any one of clauses 11-13, wherein the first opening is configured to receive the first needle spacer element (7a), and the second opening is configured to receive the second needle spacer element (7b).

23. The injection assembly as described in clause 22 when dependent on clause 20, wherein the first needle spacer element comprises a first counter needle mount configured to be engaged with the first needle mount and the second needle spacer element comprises a second counter needle mount configured to be engaged with the second needle mount.

24. The injection assembly as described in clause 20, wherein the first opening is configured to receive the first needle inserter, and the second opening is configured to receive the second needle inserter.

25. The injection assembly as described in clause 23, wherein the first needle mount and the first counter needle mount are configured to form a hook-and-loop fastener.

26. The injection assembly as described in any one of clauses 23-25, wherein the second needle mount and the second counter needle mount are adhesive.

26. The injection assembly as described in any one of clauses 14-26, wherein needle guide (13; 13′) comprises a location through-opening (11-2) defining an anatomical reference point.

27. The injection assembly as described in clause 26, wherein the needle guide is shaped to be attached to the abdomen, wherein the anatomical reference point is the umbilicus, or to a leg, wherein the anatomical reference point is the knee.

28. The injection assembly as described in clauses 26 or 27, wherein the needle guide has a symmetrical shape with respect to the location through-opening (11-2), or an asymmetrical shape with respect to the location through-opening (11-2).

29. The injection assembly as described in any one of clauses 27-28, wherein the needle guide (13′) has an inner surface and an outer surface opposite to the inner surface, wherein the outer surface has an instructional text and reversed instructional text (13c) describing an anatomical site of the anatomical reference point.

30. The injection assembly as described in any one of clauses 13-29, wherein the needle guide (13) comprises a wearable element for being worn by the user.

31. The injection assembly as described in clause 30, wherein the wearable element is a belt, a shoulder stripe, a neck stripe, a garment, a leg sleeve, or a belt clip.

32. The injection assembly as described in any one of the preceding clauses, wherein the first needle (3a) is connected to a first tube (2a; 2a′); and wherein the second needle (3b) is connected to a second tube (2b; 2b′).

33. The injection assembly as described in any one of clauses 1-31, wherein the first needle (3a) is a first tube; wherein the second needle (3b) is a second tube.

34. The injection assembly as described in clause 32 or 33, wherein the first and the second tubes (2a, 2b; 2a′, 2b′) are operably connected to a medicament delivery device.

35. The injection assembly as described in clause 34 when dependent on clause 4, wherein the needle guide comprises a distributing hub (22) fluidly connected to the medicament delivery device; and wherein the first and the second tubes (2a′, 2b′) are connected to the distributing hub (22).

36. The injection assembly as described in any one of clauses 32-35, wherein the injection assembly comprises tubing management hub (20) configured to retain the first tube (2a; 2a′) and the second tube (2b; 2b′).

37. The injection assembly as described in clause 36 when dependent on clause 13, wherein the tubing management hub (20) is attached to the needle guide.

38. The injection assembly as described in any one of the preceding clauses, wherein the injection assembly comprises a skin marker facing towards the skin of the use; wherein the skin marker is configured to mark a portion of the skin of the user that received the first predefined medicament administration.

39. The injection assembly as described in clause 31, wherein the skin marker comprises an adhesive mark or a stamp mark.

40. The injection assembly as descried in clauses 31 or 32 when dependent on clauses 11-13, wherein the first needle spacer element comprises the skin marker.

41. The injection assembly as descried in clauses 31 or 32 when dependent on clause 14, wherein the needle guide comprises the skin marker.

42. The injection assembly as described in clauses 15 or 16, the needle guide (13) comprises a rotational cover (132) selectively cover at least one of the guide surfaces (11-1a, 11-b, 11-1c) of the needle guide (13) such that the user can rotate the cover to cover the guide surface that is used.

43. The injection assembly as described in any one of preceding clauses when dependent on clause 11, wherein the first needle spacer element comprises a tube retention fastener configured to fasten a tube to the first needle spacer element; and wherein the tube is configured to establish a fluid communication between the first needle and a medicament delivery device.

44. A needle guide for medicament administration with a plurality of needles, wherein the needle guide is configured to be removably attached to the skin of a user and is configured to provide a spacing between a first needle of the plurality of needles and a second needle of the plurality of needles, comprising: a first guide surface (11-1a) and a second guide surface (11-1b), the first guide surface (11-1a) is configured to operably connected to the first needle for positioning the first needle relative to the needle guide (13), and the second guide surface (11-1b) is configured to operably connected to the second needle for positioning the second needle relative to the needle guide (13).

45. The needle guide as described in clause 44, wherein the needle guide (13) has an outer contour (11-1) which forms the first guide surface (11-1a) and the second guide surface (11-1b), the first guide surface (11-1a) being adapted to an outer contour of a first needle spacer element (7a; 7a′; 7a″; 7a′″; 7a′″) for positioning the first needle spacer element (7a; 7a′; 7a″; 7a′″; 7a′″) relative to the needle guide (13), and the second guide surface (11-1b) being adapted to an outer contour of a second needle spacer element (7b; 7b′″) for positioning the second needle spacer element (7b; 7b′″) relative to the needle guide (13).

46. The needle guide (1-2) as described in clause 45, wherein each of the first guide surface (11-1a) and the second guide surface (11-1b) is configured to mate with a respective one of the first needle spacer element (7a; 7a′; 7a″; 7a′″; 7a′″) and the second needle spacer element (7b; 7b′″).

47. The needle guide (1-3) as described in clause 46, wherein each of the first guide surface (11-1a) and the second guide surface is noncongruent with a respective one of the first needle spacer element (7a; 7a′; 7a″; 7a′″; 7a′″) and the second needle spacer element (7b; 7b′″).

48. The needle guide (1-3) as described in clause 47, wherein the first guide surface (11-1a) is colour matched with the first needle spacer element (7a; 7a′; 7a″; 7a′″; 7a′″), and the second guide surface (11-1b) is colour matched with the second needle spacer element (7b; 7b′″).

49. The needle guide as described in clause 44, wherein the needle guide (13′) has a first opening configured to receive the first needle (3a), and a second opening configured to receive the second needle (3b); wherein the first guide surface is either the inner contour of the first opening or the surface around the first opening; and wherein the second guide surface is either the inner contour of the second opening or the surface around the second opening.

50. The needle guide as described in clause 49, wherein the needle guide (13′) comprises a first needle mount adjacent to the first opening, and a second needle mount adjacent to the second opening; and wherein the first needle (3a) and the second needle (3b) are configured to be releasably mounted to the first and the second needle mounts of the needle guide (13′).

51. The needle guide as described in clause 49, wherein the needle guide (13) comprises multiple openings for receiving multiple needles respectively; wherein the needle guide (13) comprises multiple needle mounts adjacent to the multiple openings respectively; and wherein at least one needle mount of the multiple needle mounts comprises an identifying feature to differentiate the at least one needle mount from the rest of the multiple needle mounts.

52. The needle guide as described in clause 49, wherein the first opening is configured to receive a first needle spacer element (7a), and the second opening is configured to receive a second needle spacer element (7b).

53. The needle guide as described in clause 52, wherein the first needle spacer element comprises a first counter needle mount configured to be engaged with the first needle mount and the second needle spacer element comprises a second counter needle mount configured to be engaged with the second needle mount.

54. The needle guide as described in clause 49, wherein the first opening is configured to receive a first needle inserter, and the second opening is configured to receive a second needle inserter.

55. The needle guide as described in clause 50, 51, or 52, wherein the first needle mount and the first counter needle mount are configured to form a hook-and-loop fastener.

56. The needle guide as described in clause 50, 51, or 52, wherein the second needle mount and the second counter needle mount are adhesive.

57. The needle guide as described in any one of the preceding clauses, wherein needle guide (13; 13′) comprises a location through-opening (11-2) defining an anatomical reference point.

58. The needle guide as described in clause 57, wherein the needle guide is shaped to be attached to the abdomen, wherein the anatomical reference point is the umbilicus, or to a leg, wherein the anatomical reference point is the knee.

59. The needle guide as described in clause 58, wherein the needle guide has a symmetrical shape with respect to the location through-opening (11-2), or an asymmetrical shape with respect to the location through-opening (11-2).

60. The needle guide as described in any one of clauses 44-59, wherein the needle guide (13′) has an inner surface and an outer surface opposite to the inner surface, wherein the outer surface has an instructional text and reversed instructional text (13c) describing an anatomical site of the anatomical reference point.

61. The needle guide as described in any one of clauses 44-60, wherein the needle guide (13) comprises a wearable element for being worn by the user.

62. The needle guide as described in clause 61, wherein the wearable element is a belt, a shoulder stripe, a neck stripe, a garment, a leg sleeve, or a belt clip.

63. The needle guide as described in any one of clauses 44-62, wherein the needle guide comprises a distributing hub (22) fluidly connected to the medicament delivery device; and wherein a tube is connected to the distributing hub (22) and at least one of the first needle and the second needle.

64. The needle guide as described in any one of clauses 44-63, wherein the needle guide comprises tubing management hub (20) configured to retain a tube connected to the medicament delivery device.

65. The needle guide as described in clause 64, wherein the tubing management hub (20) is attached to the needle guide.

1-15. (canceled)

16. An injection assembly for medicament administration with a plurality of needles, the injection assembly comprising:
a first needle for a first predefined medicament administration;
a second needle for a second predefined medicament administration; and
a needle spacer arrangement configured to be removably attached to the skin of a user, the needle spacer arrangement being configured to provide a spacing between the first needle and the second needle,
wherein the spacing is predetermined based on at least one of characteristics of the medicament, characteristics of infusion parameters for each medicament, physiologic parameters, or combinations thereof such that the spacing is larger than a diameter of an expected first skin wheal formed subcutaneously as a result of the first predefined medicament administration and a diameter of an expected second skin wheal formed subcutaneously as a result of the second predefined medicament administration.

17. The injection assembly of claim 16, wherein the needle spacer arrangement comprises a first needle spacer element and a second needle spacer element physically separate from the first needle spacer element.

18. The injection assembly of claim 16, wherein the first needle spacer element comprises a first needle inserter configured to insert the first needle into the skin of the user, and the second needle spacer element comprises a second needle inserter configured to insert the second needle into the skin of the user.

19. The injection assembly of claim 17, wherein the first needle is mounted to the first needle spacer element and the second needle is mounted to the second needle spacer element.

20. The injection assembly of claim 16, wherein the needle spacer arrangement comprises a needle guide configured to be removably attached to the skin of the user.

21. The injection assembly of claim 20, wherein the needle guide has an outer contour which forms a first guide surface and a second guide surface, the first guide surface being adapted to an outer contour of the first needle spacer element for positioning the first needle spacer element relative to the needle guide, and the second guide surface being adapted to an outer contour of the second needle spacer element for positioning the second needle spacer element relative to the needle guide.

22. The injection assembly of claim 21, wherein each of the first guide surface and the second guide surface is configured to mate with a respective one of the first needle spacer element and the second needle spacer element.

23. The injection assembly of claim 21, wherein the needle guide has a first opening configured to receive the first needle, and a second opening configured to receive the second needle.

24. The injection assembly of claim 23, wherein the needle guide comprises a first needle mount adjacent to the first opening, and a second needle mount adjacent to the second opening, and wherein the first needle and the second needle are configured to be releasably mounted to the first and the second needle mounts of the needle guide.

25. The injection assembly of claim 24, wherein the needle guide comprises multiple openings for receiving multiple needles respectively, wherein the needle guide comprises multiple needle mounts adjacent to the multiple openings respectively, and wherein at least one needle mount of the multiple needle mounts comprises an identifying feature to differentiate the at least one needle mount from the rest of the multiple needle mounts.

26. The injection assembly of claim 23, wherein the first opening is configured to receive the first needle spacer element, and the second opening is configured to receive the second needle spacer element.

27. The injection of claim 26, wherein the first needle spacer element comprises a first counter needle mount configured to be engaged with the first needle mount and the second needle spacer element comprises a second counter needle mount configured to be engaged with the second needle mount.

28. The injection assembly of claim 27, wherein the first needle mount and the first counter needle mount are configured to form a hook-and-loop fastener.

29. The injection assembly of claim 20, wherein the needle guide comprises a wearable element for being worn by the user.

30. The injection assembly of claim 16, wherein the injection assembly comprises a skin marker facing towards the skin of the use, and wherein the skin marker is configured to mark a portion of the skin of the user that received the first predefined medicament administration.
